SEQUENCE LISTING (85 sequences)

SEQ ID NO: 1 - DNA, 20 bp, Artificial Sequence
  Synthetic
ggccggggactcggcggatc

SEQ ID NO: 2 - DNA, 20 bp, Artificial Sequence
  Synthetic
tccccggctcgacctcgttt

SEQ ID NO: 3 - DNA, 19 bp, Artificial Sequence
  Synthetic
ccagggcgcaagggagcgg

SEQ ID NO: 4 - DNA, 20 bp, Artificial Sequence
  Synthetic
tcctccgctcccttgcgccc

SEQ ID NO: 5 - DNA, 20 bp, Artificial Sequence
  Synthetic
gggggcgcgagtgatcagct

SEQ ID NO: 6 - DNA, 20 bp, Artificial Sequence
  Synthetic
cgggtttcagggctggacgg

SEQ ID NO: 7 - DNA, 20 bp, Artificial Sequence
  Synthetic
tggtccggagaaagaaggcg

SEQ ID NO: 8 - DNA, 20 bp, Artificial Sequence
  Synthetic
agcgccagagcgcgagagcg

SEQ ID NO: 9 - DNA, 19 bp, Artificial Sequence
  Synthetic
gaaggtgaaggtcggagtc

SEQ ID NO: 10 - DNA, 20 bp, Artificial Sequence
  Synthetic
gaagatggtgatgggatttc

SEQ ID NO: 11 - DNA, 20 bp, Artificial Sequence
  Synthetic
cagcaagcccagacaggtgg

SEQ ID NO: 12 - DNA, 20 bp, Artificial Sequence
  Synthetic
gcacgcggctaatcgaactc

SEQ ID NO: 13 - DNA, 20 bp, Artificial Sequence
  Synthetic
aatttggggacgagtttgtg

SEQ ID NO: 14 - DNA, 20 bp, Artificial Sequence
  Synthetic
catggtggtggacttcctct

SEQ ID NO: 15 - DNA, 20 bp, Artificial Sequence
  Synthetic
agactgccagcactttgcta

SEQ ID NO: 16 - DNA, 20 bp, Artificial Sequence
  Synthetic
gtagctccatatcctggcgg

SEQ ID NO: 17 - DNA, 16 bp, Artificial Sequence
  Synthetic
ggtgcccagcgaatgc

SEQ ID NO: 18 - DNA, 19 bp, Artificial Sequence
  Synthetic
tgatgctgtccacgatgga

SEQ ID NO: 19 - DNA, 21 bp, Artificial Sequence
  Synthetic
gctacaaggtggtgtcagggt

SEQ ID NO: 20 - DNA, 22 bp, Artificial Sequence
  Synthetic
gagccatagtacggaagcagag

SEQ ID NO: 21 - DNA, 21 bp, Artificial Sequence
  Synthetic
tctggccaaaaatgtgagcct

SEQ ID NO: 22 - DNA, 19 bp, Artificial Sequence
  Synthetic
gggtcagttagggttgggc

SEQ ID NO: 23 - DNA, 20 bp, Artificial Sequence
  Synthetic
tgcttccctgagacccagtt

SEQ ID NO: 24 - DNA, 25 bp, Artificial Sequence
  Synthetic
gatcacttctttcctttgcatcaag

SEQ ID NO: 25 - DNA, 20 bp, Artificial Sequence
  Synthetic
caaccccgcatacacctagt

SEQ ID NO: 26 - DNA, 20 bp, Artificial Sequence
  Synthetic
cgtctcgctccctcttacag

SEQ ID NO: 27 - DNA, 19 bp, Artificial Sequence
  Synthetic
aacctgcgcgagactttcc

SEQ ID NO: 28 - DNA, 20 bp, Artificial Sequence
  Synthetic
acagctggacagggagaaga

SEQ ID NO: 29 - DNA, 21 bp, Artificial Sequence
  Synthetic
ctcacctcaggtaatgggact

SEQ ID NO: 30 - DNA, 20 bp, Artificial Sequence
  Synthetic
cgtggtggtaggttccagac

SEQ ID NO: 31 - DNA, 3 bp, Artificial Sequence
  Synthetic
  misc_feature (1)..(1): n is a, c, g, or t
ngg

SEQ ID NO: 32 - DNA, 3 bp, Artificial Sequence
  Synthetic
  misc_feature (1)..(1): n is a, c, g, or t
nga

SEQ ID NO: 33 - DNA, 4 bp, Artificial Sequence
  Synthetic
  misc_feature (1)..(1): n is a, c, g, or t
  misc_feature (4)..(4): n is a, c, g, or t
ngan

SEQ ID NO: 34 - DNA, 4 bp, Artificial Sequence
  Synthetic
  misc_feature (1)..(1): n is a, c, g, or t
  misc_feature (3)..(3): n is a, c, g, or t
ngng

SEQ ID NO: 35 - DNA, 5 bp, Artificial Sequence
  Synthetic
  misc_feature (1)..(1): n is a, c, g, or t
  misc_feature (4)..(4): n is a, c, g, or t
nggng

SEQ ID NO: 36 - DNA, 7 bp, Artificial Sequence
  Synthetic
  misc_feature (1)..(2): n is a, c, g, or t
nnagaaw

SEQ ID NO: 37 - DNA, 4 bp, Artificial Sequence
  Synthetic
  misc_feature (1)..(1): n is a, c, g, or t
naar

SEQ ID NO: 38 - DNA, 5 bp, Artificial Sequence
  Synthetic
  misc_feature (1)..(2): n is a, c, g, or t
nngrr

SEQ ID NO: 39 - DNA, 6 bp, Artificial Sequence
  Synthetic
  misc_feature (1)..(2): n is a, c, g, or t
  misc_feature (6)..(6): n is a, c, g, or t
nngrrn

SEQ ID NO: 40 - DNA, 6 bp, Artificial Sequence
  Synthetic
  misc_feature (1)..(2): n is a, c, g, or t
nngrrt

SEQ ID NO: 41 - DNA, 6 bp, Artificial Sequence
  Synthetic
  misc_feature (1)..(2): n is a, c, g, or t
nngrrv

SEQ ID NO: 42 - DNA, 4107 bp, Artificial Sequence
  Synthetic
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 43 - protein, 1368 aa, Artificial Sequence [sequence omitted]

SEQ ID NO: 44 - DNA, 3158 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 45 - DNA, 3159 bp, Artificial Sequence
  Synthetic
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 46 - DNA, 3159 bp, Artificial Sequence
  Synthetic
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 47 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 48 - DNA, 3239 bp, Artificial Sequence
  Synthetic
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaa

SEQ ID NO: 49 - protein, 1053 aa, Artificial Sequence
  Synthetic
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 50 - DNA, 3159 bp, Artificial Sequence
  Synthetic
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 51 - DNA, 3159 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 52 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 53 - DNA, 3156 bp, Artificial Sequence
  Synthetic
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 54 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 55 - DNA, 7009 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 56 - DNA, 4 bp, Artificial Sequence
  Synthetic
  misc_feature (4)..(4): n is a, c, g, or t
tttn

SEQ ID NO: 57 - protein, 1497 aa, Artificial Sequence
  Synthetic
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 58 - DNA, 4491 bp, Artificial Sequence
  Synthetic
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 59 - protein, 2414 aa, Artificial Sequence
  Synthetic
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 60 - protein, 617 aa, Artificial Sequence
  Synthetic
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 61 - DNA, 20 bp, Artificial Sequence
  Synthetic
cggggctctgacattacaca

SEQ ID NO: 62 - DNA, 20 bp, Artificial Sequence
  Synthetic
gccagagtccgccctatttc

SEQ ID NO: 63 - DNA, 20 bp, Artificial Sequence
  Synthetic
tattggtcctccgctccctt

SEQ ID NO: 64 - DNA, 20 bp, Artificial Sequence
  Synthetic
gtgagcgcgatctgataggt

SEQ ID NO: 65 - DNA, 20 bp, Artificial Sequence
  Synthetic
ttgccgactttggattcgtc

SEQ ID NO: 66 - DNA, 19 bp, Artificial Sequence
  Synthetic
tccaaagggaatcccgtgc

SEQ ID NO: 67 - DNA, 20 bp, Artificial Sequence
  Synthetic
cgcagggctgaaattctggt

SEQ ID NO: 68 - DNA, 20 bp, Artificial Sequence
  Synthetic
agagccgagaaactgtcagg

SEQ ID NO: 69 - RNA, 20 nt, Artificial Sequence
  Synthetic
ggccggggacucggcggauc

SEQ ID NO: 70 - RNA, 20 nt, Artificial Sequence
  Synthetic
uccccggcucgaccucguuu

SEQ ID NO: 71 - RNA, 19 nt, Artificial Sequence
  Synthetic
ccagggcgcaagggagcgg

SEQ ID NO: 72 - RNA, 20 nt, Artificial Sequence
  Synthetic
uccuccgcucccuugcgccc

SEQ ID NO: 73 - RNA, 20 nt, Artificial Sequence
  Synthetic
gggggcgcgagugaucagcu

SEQ ID NO: 74 - RNA, 20 nt, Artificial Sequence
  Synthetic
cggguuucagggcuggacgg

SEQ ID NO: 75 - RNA, 20 nt, Artificial Sequence
  Synthetic
ugguccggagaaagaaggcg

SEQ ID NO: 76 - RNA, 20 nt, Artificial Sequence
  Synthetic
agcgccagagcgcgagagcg

SEQ ID NO: 77 - DNA, 20 bp, Artificial Sequence
  Synthetic
gatccgccgagtccccggcc

SEQ ID NO: 78 - DNA, 20 bp, Artificial Sequence
  Synthetic
aaacgaggtcgagccgggga

SEQ ID NO: 79 - DNA, 19 bp, Artificial Sequence
  Synthetic
ccgctcccttgcgccctgg

SEQ ID NO: 80 - DNA, 20 bp, Artificial Sequence
  Synthetic
gggcgcaagggagcggagga

SEQ ID NO: 81 - DNA, 20 bp, Artificial Sequence
  Synthetic
agctgatcactcgcgccccc

SEQ ID NO: 82 - DNA, 20 bp, Artificial Sequence
  Synthetic
ccgtccagccctgaaacccg

SEQ ID NO: 83 - DNA, 20 bp, Artificial Sequence
  Synthetic
cgccttctttctccggacca

SEQ ID NO: 84 - DNA, 20 bp, Artificial Sequence
  Synthetic
cgctctcgcgctctggcgct

SEQ ID NO: 85 - DNA, 83 bp, Artificial Sequence
  Synthetic
gttttagagctagaaatagcaagttaaaataaggctagtccgttatcaacttgaaaaagtggcaccgagtcggtgcttttttt

(19) United States
(12) Patent Application Publication
(10) Pub. No.: US 2022/0305141 A1
(43) Pub. Date: Sep. 29, 2022

(54) SKELETAL MYOBLAST PROGENITOR CELL LINEAGE SPECIFICATION BY CRISPR/CAS9-BASED TRANSCRIPTIONAL ACTIVATORS

(71) Applicant: Duke University, Durham, NC (US)

(72) Inventors: Charles A. Gersbach, Chapel Hill, NC (US); Jennifer Kwon, Durham, NC (US)

(21) Appl. No.: 17/636,754

(22) PCT Filed: Aug. 19, 2020
(86) PCT No.: PCT/US20/47080

§ 371 Date: Feb. 18, 2022

Related U.S. Application Data
(60) Provisional application No. 62/888,916, filed on Aug. 19, 2019.
(60) Provisional application No. 62/968,743, filed on Jan. 31, 2020.

(51) Int. Cl. A61K 48/00; A61K 35/34; C07K 14/315; C07K 14/47; C12N 15/113; C12N 15/63; C12N 15/90; C12N 9/22

(52) U.S. Cl. CPC A61K 48/0058; A61K 35/34; C07K 14/315; C07K 14/4702; C12N 15/113; C12N 15/63; C12N 15/907; C12N 9/22; C12N 2310/20; C12N 2740/16043; A61K 38/00

(57) ABSTRACT

Disclosed herein are methods and systems for increasing expression of Pax7, methods of activating endogenous myogenic transcription factor Pax7 in a cell, methods of differentiating a stem cell into a skeletal muscle progenitor cell, as well as compositions and methods for treating a subject in need of regenerative muscle progenitor cells. The compositions and methods may include a Cas9-based transcriptional activator protein and at least one guide RNA (gRNA) targeting Pax7.

### CROSS-REFERENCE TO RELATED APPLICATIONS

This application claims priority to U.S. Provisional Patent Application No. 62/888,916, filed Aug. 19, 2019, and U.S. Provisional Patent Application No. 62/968,743, filed Jan. 31, 2020, each of which is incorporated herein by reference in its entirety.

### STATEMENT REGARDING FEDERALLY SPONSORED RESEARCH

This invention was made with government support under grant 1DP2-OD008586 and 1R01DA036865 awarded by the National Institutes of Health. The government has certain rights in the invention.

### FIELD

This disclosure relates to compositions and methods for increasing the expression of Pax7 in stem cells, inducing differentiation of a stem cell into a skeletal muscle progenitor cell, and using these skeletal muscle progenitor cells to regenerate damaged muscle tissue.

### INTRODUCTION

Human pluripotent stem cells (hPSCs) are a promising cell source for regenerative medicine, disease modeling, and drug discovery in pathologies of muscle disease. Directed differentiation of hPSCs into skeletal muscle cells can be achieved via stepwise small molecule-based protocols or ectopic expression of transgenes. While having the benefit of being transgene-free, small molecule-based protocols tend to be relatively lengthy, inefficient, and lack the scalability required for cell therapy or drug screening applications. Transgene-based approaches rely on overexpression of key myogenic transcription factors, including Pax3, Pax7, and MyoD. These protocols are highly efficient in yielding populations of myogenic cells, and they do so more rapidly than transgene-free methods. Generation of satellite cells, such as the skeletal muscle stem cell population, is particularly appealing for myogenic cell therapies. Although satellite cells can robustly regenerate damaged muscles in vivo, they cannot be isolated and expanded ex vivo without relinquishing their stemness, resulting in loss of engraftment capabilities. As such, the generation of functional Pax7+ satellite cells from hPSCs has been attempted by pairing various differentiation protocols with exogenous Pax7 cDNA overexpression. There is a need for alternative methods for generating populations of myogenic cells.

### SUMMARY

In an aspect, the disclosure relates to a guide RNA (gRNA) molecule targeting Pax7 or a promoter or regulatory element of the Pax7 gene. The gRNA may comprise a polynucleotide sequence corresponding to at least one of SEQ ID NOs: 1-8 or 69-76, or a variant thereof.

In a further aspect, the disclosure relates to a DNA targeting system for increasing expression of Pax7. The DNA targeting system may comprise at least one gRNA that binds and targets a Pax7 gene or a portion thereof. In some embodiments, the at least one gRNA comprises a polynucleotide sequence corresponding to at least one of SEQ ID NOs: 1-8 or 69-76, or a variant thereof.

In some embodiments, the DNA targeting system further includes a Clustered Regularly Interspaced Short Palindromic Repeats associated (Cas) protein or a fusion protein, wherein the fusion protein comprises two heterologous polypeptide domains, wherein the first polypeptide domain comprises a Cas protein, a zinc finger protein, or a TALE protein, and the second polypeptide domain has transcription activation activity. In some embodiments, the Cas protein comprises a Streptococcus pyogenes Cas9 molecule, or a variant thereof. In some embodiments, the fusion protein comprises VP64-dCas9-VP64 (^{VP64}dCas9^{VP64}). In some embodiments, the Cas protein comprises a Cas9 that recognizes a Protospacer Adjacent Motif (PAM) of NGG (SEQ ID NO: 31), NGA (SEQ ID NO: 32), NGAN (SEQ ID NO: 33), or NGNG (SEQ ID NO: 34).

Another aspect of the disclosure provides an isolated polynucleotide sequence comprising a gRNA molecule as disclosed herein.

Another aspect of the disclosure provides an isolated polynucleotide sequence encoding a DNA targeting system as disclosed herein.

Another aspect of the disclosure provides a vector comprising an isolated polynucleotide sequence as disclosed herein.

Another aspect of the disclosure provides a vector encoding a gRNA molecule as disclosed herein and a Clustered Regularly Interspaced Short Palindromic Repeats associated (Cas) protein.

Another aspect of the disclosure provides a cell comprising a gRNA as disclosed herein, a DNA targeting system as disclosed herein, an isolated polynucleotide sequence as disclosed herein, or a vector as disclosed herein, or a combination thereof.

Another aspect of the disclosure provides a pharmaceutical composition comprising a gRNA as disclosed herein, a DNA targeting system as disclosed herein, an isolated polynucleotide sequence as disclosed herein, a vector as disclosed herein, or a cell as disclosed herein, or a combination thereof.

Another aspect of the disclosure provides a method of activating endogenous myogenic transcription factor Pax7 in a cell. The method may include administering to the cell a gRNA as disclosed herein, a DNA targeting system as disclosed herein, an isolated polynucleotide sequence as disclosed herein, or a vector as disclosed herein.

Another aspect of the disclosure provides a method of differentiating a stem cell into a skeletal muscle progenitor cell. The method may include administering to the stem cell a gRNA as disclosed herein, a DNA targeting system as disclosed herein, an isolated polynucleotide sequence as disclosed herein, or a vector as disclosed herein.

In some embodiments, endogenous expression of Pax7 mRNA is increased in the skeletal muscle progenitor cell. In some embodiments, the expression of Myf5, MyoD, MyoG, or a combination thereof, is increased in the skeletal muscle progenitor cell. In some embodiments, the stem cell is induced into myogenic differentiation. In some embodiments, the skeletal muscle progenitor cell maintains Pax7 expression after at least about 6 passages.

Another aspect of the disclosure provides a method of treating a subject in need thereof. The method may include administering to the subject a cell as disclosed herein.

In some embodiments, the level of dystrophin+ fibers in the subject is increased.

In some embodiments, muscle regeneration in the subject is increased.

The disclosure provides for other aspects and embodiments that will be apparent in light of the following detailed description and accompanying figures.

### BRIEF DESCRIPTION OF THE DRAWINGS

FIGS. 1A-1G. Generation of myogenic progenitors from hPSCs via VP64-dCas9-VP64-mediated activation of endogenous PAX7. (FIG. 1A) Schematic of hPSC myogenic differentiation with small molecules and lentiviral activation of PAX7. (FIG. 1B) The lentiviral constructs used for the gRNA and inducible VP64-dCas9-VP64 and PAX7 cDNA expression. (FIG. 1C) Representative phase-contrast images showing morphological changes during the first 10 days of differentiation. Scale bar=200 μm. (FIG. 1D) RNA was harvested at day 0 and day 2 for qRT-PCR analysis of mesodermal markers. Results are expressed as fold change over day 0 (mean t SEM, n=3 independent replicates). (FIG. 1E) Representative FACS plot at day 14 when VP64-dCas9-VP64-2a-mCherry+ cells were sorted for expansion. (FIG. 1F) Representative immunostaining of PAX7 at 5 days post-sort. Scale bar=100 μm. (FIG. 1G) Growth of purified myogenic progenitors derived from iPSC differentiation during post-sort expansion phase was monitored over 2 weeks. Fold-growth over two weeks was significantly greater in VP64-dCas9-VP64-treated cells compared to PAX7 cDNA-treated cells. P value determined by one-way ANOVA followed by Tukey's post hoc test (mean t SEM, n=3 independent replicates).

FIGS. 2A-2F. Characterization of myogenic progenitors derived from iPSCs via VP64-dCas9-VP64-mediated activation of endogenous PAX7 or exogenous PAX7 cDNA expression. (FIG. 2A) Relative amounts of total PAX7 mRNA was determined by qRT-PCR using primers complementary to sequences present in the gene body. (FIG. 2B) Endogenous PAX7 mRNA was detected using primers complementary to sequences in the 3′ UTR of either isoforms PAX7-A or PAX7-B. (FIG. 2C) The mRNA expression levels of myogenic markers MYF5, MYOD, and MYOG during the expansion phase. (FIG. 2D) Immunofluorescence staining of early and mature myogenic markers MYF5, MYOD, and MYOG, and myosin heavy chain (MHC). (FIG. 2E) Representative FACS analysis of CD29 and CD56 surface marker expression during the expansion phase. (FIG. 2F) Mean fluorescence intensity (MFI) of CD56 staining intensity across treatments. All P values were determined by one-way ANOVA followed by Tukey's post hoc test (mean t SEM, n=3 independent replicates).

FIGS. 3A-3C. Transplantation of VP64-dCas9-VP64-generated myogenic progenitors into immunodeficient mice demonstrates in vivo regenerative potential. (FIG. 3A) Detection of human-derived fibers in VP64-dCas9-VP64-treated cells 1 month after intramuscular injection of 5×10⁵ differentiated iPSCs into NSG mice pre-injured with BaCl₂. Sections are stained with human-specific dystrophin and lamin A/C antibodies to mark donor-derived fibers and nuclei. Scale bar=100 μm. (FIG. 3B) Quantification of human dystrophin+ fibers in the section with highest number of dystrophin+ fibers in each muscle. *p<0.05 determined by student's t-test compared to control (mean t SEM, n=3 mice). (FIG. 3C) Identification of donor-derived satellite cells expressing PAX7 and human-specific lamin A/C, and residing adjacent to the basal lamina as indicated by laminin staining. Scale bar=25 μm.

FIGS. 4A-4D. Induction of endogenous PAX7 expression is sustained after multiple passages and dox withdrawal. (FIG. 4A) Representative immunostaining of PAX7 and MHC in differentiated iPSCs after 4 passages in the presence of dox. Scale bar=200 μm. (FIG. 4B) Representative immunostaining of PAX7 and myosin heavy chain (MHC) after inducing differentiation by dox withdrawal for 7 days. Scale bar=200 μm. (FIG. 4C) Quantification of PAX7+ nuclei after 0 passages and after an average of 4 additional passages with dox or after dox withdrawal (mean t SEM, n=3 independent experiments). (FIG. 4D) Representative immunostaining of the FLAG epitope for VP64-dCas9-VP64 after dox withdrawal for 7 days. Scale bar=100 μm.

FIGS. 5A-6D. VP64-dCas9-VP64 leads to sustained PAX7 expression and stable chromatin remodeling at target locus. (FIG. 5A) Human genomic track spanning the PAX7 TSS region depicting H3K4me3 and H3K27ac enrichment in human skeletal muscle myoblast (HSMM). Data from ENCODE (GEO:GSM733637; GEO:GSM733755). Black bars indicate ChIP-qPCR target regions. (FIG. 5B) Targeted activation of endogenous PAX7 induced significant enrichment of H3K4me3 and H3K27ac around the TSS in the presence of dox in proliferation conditions. (FIG. 5C) Enrichment of histone marks is sustained after 15 days in the absence of dox in proliferation conditions (mean t SEM, n=3 independent replicates). (FIG. 5D) An N-terminal FLAG epitope tag was used to verify depletion of VP64-dCas9-VP64 after 15 days without dox, which was concomitant with sustained PAX7 protein expression.

FIGS. 6A-6E. Identification of endogenous vs. exogenous PAX7-induced global transcriptional changes. (FIG. 6A) An expression heatmap of sample-to-sample distances in the matrix using the whole gene expression profiles among the 4 groups and their replicates. (FIG. 6B) Heatmap showing differential expression of top 200 variable genes between all 4 groups after filtering genes with low read counts. The color bar indicates z-score. (FIG. 6C) Venn diagram of genes overexpressed in each group relative to gRNA only (fold-change >2 and padj <0.05) (FIG. 6D) GO Biological process terms of shared genes between the 3 groups derived from the Venn diagram in FIG. 4C. Term list was generated using Enrichr; P-values were computed using the Fisher exact test. (FIG. 6E) Expression profiles of select premyogenic, myogenic, and satellite cell marker genes from RNA-seq data (mean t SEM, n=3 independent replicates). TPM: Transcripts Per Million.

FIGS. 7A-7C. Screening gRNAs for PAX7 activation with VP64-dCas9-VP64, related to FIGS. 1A-1G. (FIG. 7A) gRNA target sites relative to genome browser position of the human PAX7 gene. (FIG. 7B) Cells expressing VP64-dCas9-VP64 were treated for two days with CHIRON99021 and lipofected with PAX7-targeting gRNAs. Cells were harvested for qRT-PCR analysis after 6 days. gRNA 3, 4, 5 and 8 significantly upregulated PAX7 compared to mock transfection, but were not significantly different from each other. (FIG. 7C) Lentiviral transduction of gRNAs in paraxial mesoderm cells expressing P64-dCas9-VP64 and gRNAs for 1 week. gRNA 4 significantly outperformed the other gRNAs. P-values were determined by one-way ANOVA followed by Tukey's post hoc test; p<0.05 (mean t SEM, n=3 independent replicates).

FIGS. 8A-8J. Characterization and transplantation of myogenic progenitors derived from H9 ESCs via VP64dCas9VP64-mediated activation of endogenous PAX7 or exogenous PAX7 cDNA expression, related to FIGS. 2A-2F and FIGS. 3A-3C. (FIG. 8A) Representative immunostaining of PAX7 at 5 days postsort. Scale bar=100 μm. (FIG. 8B) Growth curve of purified myogenic progenitors during post-sort expansion phase was monitored over 2 weeks. (FIG. 8C) Relative amount of total PAX7 mRNA was determined by qRT-PCR using primers complementary to sequences present in the gene body. (FIG. 8D) Endogenous PAX7 mRNA was detected using primers complementary to sequencing in the 3′ UTR of either PAX7-A or PAX7-B isoforms. (FIG. 8E) The mRNA expression levels of myogenic markers MYF5, MYOD, and MYOG during the expansion phase. (FIG. 8F) Representative FACS analysis of CD29 and CD56 surface marker expression during the expansion phase. (FIG. 8G) Mean fluorescence intensity (MFI) of CD56 staining intensity across treatments. (FIG. 8H) Representative immunostaining of PAX7 and MHC in differentiated H9 ESCs after 4 passages in the presence of dox. Scale bar=200 μm. (FIG. 8I) Detection of human-derived fibers in VP64dCas9VP64-treated cells 1 month after intramuscular injection of 5×10⁵ differentiated ESCs into NSG mice pre-injured with BaCl2. Sections are stained with human-specific dystrophin and lamin A/C antibodies to mark donor-derived fibers and nuclei. Scale bar=100 μm. (FIG. 8J) Identification of donor-derived satellite cells expressing PAX7 and human specific lamin A/C. All P values were determined by one-way ANOVA followed by Tukey's post hoc test (mean t SEM, n=3 independent replicates). Scale bar=25 μm.

FIGS. 9A-9E. RNA-seq analysis, related to FIGS. 6A-6E. (FIG. 9A) Multidimensional scaling (MDS) of the top 500 differentially expressed genes. (FIG. 9B) Heatmap showing differential expression of top 50 variable genes between the 3 PAX7-expressing groups. The color bar indicates z-score. (FIG. 9C) Expression profile from selected genes overexpressed in response to cDNA encoding PAX7-A from RNA-seq (mean t SEM, n=3 independent replicates). (FIG. 9D) GO biological process terms for genes specifically enriched in cells treated with VP64dCas9VP64+gRNA, PAX7-A cDNA, or PAX7-B cDNA, corresponding to Venn diagram in FIG. 4C. (FIG. 9E) Additional expression profiles of known satellite cell surface markers.

### DETAILED DESCRIPTION

Various DNA targeting systems and methods of use thereof are disclosed herein and may include, for example, a DNA targeting system using CRISPR/Cas, zinc fingers, or TALEs.

Advances in genome engineering technologies have established the type II clustered regularly spaced short palindromic repeat (CRISPR)/Cas9 system as a programmable transcriptional regulator capable of targeted activation or repression of endogenous genes. Mutations to the catalytic residues of the Cas9 protein results in a nuclease-null Cas9 (dCas9) that can be fused to various effector domains to exert their function on precise genomic loci defined by the guide RNA (gRNA). For example, fusion of dCas9 to the transactivation domain VP64 can potently activate genes in their native chromosomal context when gRNAs are designed at target gene promoters. In contrast to ectopic expression of transgenes, activation of endogenous genes facilitates chromatin remodeling and induction of autonomously maintained gene networks. Targeting endogenous genes can also capture the full complexity of transcript isoforms, mRNA localization, and other effects of non-coding regulatory elements, which may be critical for proper cellular reprogramming. Cellular reprogramming may be achieved with CRISPR/Cas9-based transcriptional regulators in the context of somatic cell reprogramming as well as directed differentiation of pluripotent stem cells into various cell types. However, prior to the work detailed herein, there has not been demonstration of differentiation of hPSCs with CRISPR/Cas9-based transcriptional activators to generate cells capable of in vivo transplantation, engraftment, and tissue regeneration, or any attempt to generate myogenic progenitor cells via activation of the endogenous Pax7 gene.

Engineered CRISPR/Cas9-based transcriptional activators can potently and specifically activate endogenous fate-determining genes to direct differentiation of pluripotent stem cells. As detailed herein, VP64-dCas9-VP64 was used to activate the endogenous myogenic transcription factor, Pax7, to directly reprogram human pluripotent stem cells and direct differentiation of them into skeletal muscle progenitors in both human ES and iPS cells. The functional skeletal muscle progenitor cells can be induced to differentiate in vitro and can also participate in regeneration of damaged muscles in vivo when transplanted into mice. Compared to the exogenous overexpression of Pax7 cDNA, endogenous activation results in the generation of more proliferative myogenic progenitors that can maintain Pax7 expression over multiple passages in serum-free conditions while maintaining the capacity for terminal myogenic differentiation. Transplantation of myogenic progenitors derived from endogenous activation of Pax7 into immunodeficient mice resulted in a greater number of human dystrophin+ myofibers compared to exogenous Pax7 overexpression. The results detailed herein also reveal functional differences between myogenic progenitors generated via CRISPR-based endogenous activation of Pax7 and exogenous Pax7 cDNA overexpression. These studies demonstrate the utility of CRISPR/Cas9-based transcriptional activators for myogenic progenitor cell differentiation and their potential for cell therapy and musculoskeletal regenerative medicine. The methods of these studies may be applied using any DNA binding domain, such as a zinc finger protein or a TALE protein similarly to a Cas protein.

Described herein are systems for increasing expression of Pax7, which may include a Cas9 protein such as VP64-dCas9-VP64, and at least one guide RNA (gRNA) targeting Pax7 or a promoter or regulatory element of the Pax7 gene. Further provided herein are methods of activating endogenous myogenic transcription factor Pax7 in a cell, methods of differentiating a stem cell into a skeletal muscle progenitor cell, and methods of treating a subject in need thereof. The methods may include administering to the cell or subject the system for increasing expression of Pax7, or administering a cell transduced or transfected by the system.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a nuclease target region that is recognized and bound by the nuclease.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimize.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice. A control may be an subject or cell without the system as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Fusion protein” as used herein refers to a chimeric protein created through the translation of two or more joined genes that originally coded for separate proteins. The translation of the fusion gene results in a single polypeptide with functional properties derived from each of the original separate proteins.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Genome editing” or “gene editing” as used herein refers to changing a gene. Genome editing may include correcting or restoring a mutant gene. Genome editing may include knocking out a gene, such as a mutant gene or a normal gene. Genome editing may be used to treat disease or enhance muscle repair by changing the gene of interest.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded, or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to after the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a DNA targeting system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Spacers” and “spacer region” as used interchangeably herein refers to the region within a TALE or zinc finger target region that is between, but not a part of, the binding regions for two TALEs or zinc finger proteins.

“Subject” or “patient” as used herein can mean an animal that wants or is in need of the herein described compositions or methods. The subject may be a human or a non-human. The subject may be any vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Transcription activator-like effector” or “TALE” refers to a protein structure that recognizes and binds to a particular DNA sequence. The “TALE DNA-binding domain” refers to a DNA-binding domain that includes an array of tandem 33-35 amino acid repeats, also known as RVD modules, each of which specifically recognizes a single base pair of DNA. RVD modules may be arranged in any order to assemble an array that recognizes a defined sequence. A binding specificity of a TALE DNA-binding domain is determined by the RVD array followed by a single truncated repeat of 20 amino acids. “Repeat variable diresidue” or “RVD” refers to a pair of adjacent amino acid residues within a DNA recognition motif (also known as “RVD module”), which includes 33-35 amino acids, of a TALE DNA-binding domain. The RVD determines the nucleotide specificity of the RVD module. RVD modules may be combined to produce an RVD array. The “RVD array length” as used herein refers to the number of RVD modules that corresponds to the length of the nucleotide sequence within the TALEN target region that is recognized by a TALEN, i.e., the binding region A TALE DNA-binding domain may have 12 to 27 RVD modules, each of which contains an RVD and recognizes a single base pair of DNA. Specific RVDs have been identified that recognize each of the four possible DNA nucleotides (A, T, C, and G). Because the TALE DNA-binding domains are modular, repeats that recognize the four different DNA nucleotides may be linked together to recognize any particular DNA sequence. These targeted DNA-binding domains may then be combined with catalytic domains to create functional enzymes, including artificial transcription factors, methyltransferases, integrases, nucleases, and recombinases.

“Target gene” as used herein refers to any nucleotide sequence encoding a known or putative gene product. The target gene may be a mutated gene involved in a genetic disease. In certain embodiments, the target gene is Pax7 or a transcription factor for Pax7 or a regulatory element for Pax7.

“Target region” as used herein refers to the region of the target gene to which the CRISPR/Cas9-based gene editing system is designed to bind.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Treatment” or “treating,” when referring to protection of a subject from a disease, means suppressing, repressing, ameliorating, or completely eliminating the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art (Kyte et al., J. Mol. Bol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid. For example, the vector may encode a Cas9 protein and at least one gRNA molecule.

“Zinc finger” as used herein refers to a protein that recognizes and binds to DNA sequences. The zinc finger domain is the most common DNA-binding motif in the human proteome. A single zinc finger contains approximately 30 amino acids, and the domain typically functions by binding 3 consecutive base pairs of DNA via interactions of a single amino acid side chain per base pair.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Pax7

Pax7 (paired box gene 7) is a protein that acts as a myogenic transcription factor. Pax7 may be factor in the expression of neural crest markers such as, for example, Slug, Sox9, Sox10, and HNK-1. Pax7 may be expressed in the palatal shelf of the maxilla, Meckel's cartilage, mesencephalon, nasal cavity, nasal epithelium, nasal capsule, and pons. Pax7 can bind to DNA as a heterodimer with Pax3. Pax7 may also interact with PAXBP1 and/or DAXX.

Pax7 is a transcription factor that plays a role in myogenesis through regulation of muscle precursor cells proliferation. Skeletal muscle growth and regeneration are attributed to satellite cells, which are muscle stem cells resident beneath the basal lamina that surrounds each myofibre. Quiescent satellite cells express the transcription factor Pax7, and when activated, the quiescent satellite cells may coexpress Pax7 with MyoD. Most cells may then proliferate, downregulate Pax7, and differentiate. By contrast, other cells may maintain expression of Pax7 but lose expression of MyoD, and return to a state resembling quiescence. Upon expression or activation of Pax7 in a stem cell, the stem cell may differentiate into a skeletal muscle progenitor cell. The stem cell may be, for example, an induced pluripotent stem cell (iPSC) or an embryonic stem cell (ESC). The stem cell may be induced into myogenic differentiation. In some embodiments, expression or activation of Pax7 results in expression of Myf5, MyoD, MyoG, or a combination thereof. In some embodiments, expression or activation of Pax7 results in muscle regeneration. In some embodiments, expression or activation of Pax7 results in an increase of muscle stem cells, which may contribute to dystrophin+ fibers.

### 3. CRISPR/Cas-Based Gene Editing System

Provided herein are genetic constructs for genome editing, genomic alteration, or altering gene expression of a gene, for example, a gene encoding Pax7. The genetic constructs include at least one gRNA that targets a gene sequence. The disclosed gRNAs can be included in a CRISPR/Cas9-based gene editing system to target regions in the Pax7 gene, or a promoter or regulatory element of the Pax7 gene, causing activation of endogenous expression of Pax7.

A CRISPR/Cas-based gene editing system may be specific for the Pax7 gene, or a promoter or regulatory element of the Pax7 gene. The CRISPR/Cas-based gene editing system may be a CRISPR/Cas9-based gene editing system specific for the Pax7 gene, or a promoter or regulatory element of the Pax7 gene. “Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a ‘memory’ of past exposures. A Cas protein, such as a Cas9 protein, forms a complex with the 3′ end of the sgRNA (also referred interchangeably herein as “gRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and Ill effector systems) are known. The Type II effector system carries out targeted DNA double-strand break in four sequential steps, using a single effector enzyme such as Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements. The Streptococcus pyogenes CRISPR system may have the PAM sequence for this Cas9 (SpCas9) as 5′-NRG-3′, where R is either A or G. and characterized the specificity of this system in human cells. A unique capability of the CRISPR/Cas9-based gene editing system is the straightforward ability to simultaneously target multiple distinct genomic loci by co-expressing a single Cas9 protein with two or more sgRNAs. For example, the S. pyogenes Type II system naturally prefers to use an “NGG” sequence, where “N” can be any nucleotide, but also accepts other PAM sequences, such as “NGG” in engineered systems (Hsu et al., Nature Biotechnology 2013 doi:10.1038/nbt.2647). Similarly, the Cas9 derived from Neisseria meningitidis (NmCas9) normally has a native PAM of NNNNGATT, but has activity across a variety of PAMs, including a highly degenerate NNNNGNNN PAM (Esvelt et al. Nature Methods 2013 doi:10.1038/nmeth.2681).

A Cas9 molecule of S. aureus recognizes the sequence motif NNGRR (R=A or G) (SEQ ID NO: 38) and directs cleavage of a target nucleic acid sequence 1 to 10, e.g., 3 to 5, bp upstream from that sequence. In certain embodiments, a Cas9 molecule of S. aureus recognizes the sequence motif NNGRRN (R=A or G) (SEQ ID NO: 39) and directs cleavage of a target nucleic acid sequence 1 to 10, e.g., 3 to 5, bp upstream from that sequence. In certain embodiments, a Cas9 molecule of S. aureus recognizes the sequence motif NNGRRT (R=A or G) (SEQ ID NO: 40) and directs cleavage of a target nucleic acid sequence 1 to 10, e.g., 3 to 5, bp upstream from that sequence. In certain embodiments, a Cas9 molecule of S. aureus recognizes the sequence motif NNGRRV (R=A or G) (SEQ ID NO: 41) and directs cleavage of a target nucleic acid sequence 1 to 10, e.g., 3 to 5, bp upstream from that sequence. In the aforementioned embodiments, N can be any nucleotide residue, e.g., any of A, G, C, or T. Cas9 molecules can be engineered to alter the PAM specificity of the Cas9 molecule.

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in genome editing and treating genetic diseases. The CRISPR/Cas9-based engineered systems can be designed to target any gene, including genes involved in a genetic disease, aging, tissue regeneration, or wound healing. The CRISPR/Cas9-based gene editing systems can include a Cas9 protein or Cas9 fusion protein and at least one gRNA. In certain embodiments, the system comprises two gRNA molecules. The Cas9 fusion protein may, for example, include a domain that has a different activity that what is endogenous to Cas9, such as a transactivation domain.

The target gene (e.g., the Pax7 gene, or a regulatory element of the Pax7 gene) can be involved in differentiation of a cell or any other process in which activation of a gene can be desired, or can have a mutation such as a frameshift mutation or a nonsense mutation. In some embodiments, the target or target gene includes a regulatory element of the Pax7 gene. The CRISPR/Cas9-based gene editing system may or may not mediate off-target changes to protein-coding regions of the genome. The CRISPR/Cas9-based gene editing system may bind and recognize a target region. The targeted gene may be the Pax7 gene.

a. Cas Protein

The CRISPR/Cas-based gene editing system can include a Cas protein or a Cas fusion protein. In some embodiments, the Cas protein is a Cas12 protein (also referred to as Cpf1), such as a Cas12a protein. The Cas12 protein can be from any bacterial or archaea species, including, but not limited to, Francisella novicida, Acidaminococcus sp., Lachnospiraceae sp., and Prevotella sp. In some embodiments, the Cas protein is a Cas9 protein. Cas9 protein is an endonuclease that may cleave nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., cycliphilus denitritcans, Aminomonas paucivorans, Bacillus cereus. Bacillus smithii, Bacillus thuringiensis, Bacteroides sp., Blastopirellula manna, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterum dolichum, gamma proteobacterum, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris. Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). In certain embodiments, the Cas9 molecule is a Staphylococcus aureus Cas9 molecule (also referred herein as “SaCas9”).

A Cas molecule or a Cas fusion protein can interact with one or more gRNA molecules and, in concert with the gRNA molecule(s), can localize to a site which comprises a target domain, and in certain embodiments, a PAM sequence. The ability of a Cas molecule or a Cas fusion protein to recognize a PAM sequence can be determined, e.g., using a transformation assay as known in the art.

In certain embodiments, the ability of a Cas molecule or a Cas fusion protein to interact with and cleave a target nucleic acid is protospacer-adjacent motif (PAM) sequence dependent. A PAM sequence is a sequence in the target nucleic acid. In certain embodiments, cleavage of the target nucleic acid occurs upstream from the PAM sequence. Cas molecules from different bacterial species can recognize different sequence motifs (e.g., PAM sequences). In certain embodiments, a Cas12 molecule of Francisella novicida recognizes the sequence motif TTTN (SEQ ID NO: 56). In certain embodiments, a Cas9 molecule of S. pyogenes recognizes the sequence motif NGG and directs cleavage of a target nucleic acid sequence 1 to 10, e.g., 3 to 5, bp upstream from that sequence. In certain embodiments, a Cas9 molecule of S. thermophilus recognizes the sequence motif NGGNG (SEQ ID NO: 35) and/or NNAGAAW (W=A or T) (SEQ ID NO: 36) and directs cleavage of a target nucleic acid sequence 1 to 10, e.g., 3 to 5, bp upstream from these sequences. In certain embodiments, a Cas9 molecule of S. mutans recognizes the sequence motif NGG (SEQ ID NO: 31) and/or NAAR (R=A or G) (SEQ ID NO: 37) and directs cleavage of a target nucleic acid sequence 1 to 10, e.g., 3 to 5 bp, upstream from this sequence. In certain embodiments, a Cas9 molecule of S. aureus recognizes the sequence motif NNGRR (R=A or G) (SEQ ID NO: 38) and directs cleavage of a target nucleic acid sequence 1 to 10, e.g., 3 to 5, bp upstream from that sequence. In certain embodiments, a Cas9 molecule of S. aureus recognizes the sequence motif NNGRRN (R=A or G) (SEQ ID NO: 39) and directs cleavage of a target nucleic acid sequence 1 to 10, e.g., 3 to 5, bp upstream from that sequence. In certain embodiments, a Cas9 molecule of S. aureus recognizes the sequence motif NNGRRT (R=A or G) (SEQ ID NO: 40) and directs cleavage of a target nucleic acid sequence 1 to 10, e.g., 3 to 5, bp upstream from that sequence. In certain embodiments, a Cas9 molecule of S. aureus recognizes the sequence motif NNGRRV (R=A or G; V=A or C or G) (SEQ ID NO: 41) and directs cleavage of a target nucleic acid sequence 1 to 10, e.g., 3 to 5, bp upstream from that sequence. In the aforementioned embodiments, N can be any nucleotide residue, e.g., any of A, G, C, or T. Cas9 molecules can be engineered to alter the PAM specificity of the Cas9 molecule.

In certain embodiments, the vector encodes at least one Cas9 molecule that recognizes a Protospacer Adjacent Motif (PAM) of either NNGRRT (SEQ ID NO: 40) or NNGRRV (SEQ ID NO: 41). In certain embodiments, the at least one Cas9 molecule is an S. aureus Cas9 molecule. In certain embodiments, the at least one Cas9 molecule is a mutant S. aureus Cas9 molecule.

The Cas protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence include: D10A, E762A, H840A, N854A, N863A, and/or D986A. Exemplary mutations with reference to the S. aureus Cas9 sequence include D10A and N580A. In certain embodiments, the Cas9 molecule is a mutant S. aureus Cas9 molecule. In some embodiments, the dCas9 is a Cas9 molecule that includes at least two mutations selected from D10A, E762A, H840A, N854A, N863A, and/or D986A, with reference to the S. pyogenes Cas9 sequence. In some embodiments, the Cas protein is a dCas9 protein. In some embodiments, the Cas protein is a dCas12 protein.

In certain embodiments, the mutant S. aureus Cas9 molecule comprises a D10A mutation. The nucleotide sequence encoding this mutant S. aureus Cas9 is set forth in SEQ ID NO: 50.

In certain embodiments, the mutant S. aureus Cas9 molecule comprises a N580A mutation. The nucleotide sequence encoding this mutant S. aureus Cas9 molecule is set forth in SEQ ID NO: 51.

A polynucleotide encoding a Cas molecule can be a synthetic polynucleotide. For example, the synthetic polynucleotide can be chemically modified. The synthetic polynucleotide can be codon optimized, e.g., at least one non-common codon or less-common codon has been replaced by a common codon. For example, the synthetic polynucleotide can direct the synthesis of an optimized messenger mRNA, e.g., optimized for expression in a mammalian expression system, e.g., described herein.

Additionally or alternatively, a nucleic acid encoding a Cas molecule or Cas polypeptide may comprise a nuclear localization sequence (NLS). Nuclear localization sequences are known in the art. An exemplary codon optimized nucleic acid sequence encoding a Cas9 molecule of S. pyogenes is set forth in SEQ ID NO: 42. The corresponding amino acid sequence of an S. pyogenes Cas9 molecule is set forth in SEQ ID NO: 43.

Exemplary codon optimized nucleic acid sequences encoding a Cas9 molecule of S. aureus, and optionally containing nuclear localization sequences (NLSs), are set forth in SEQ ID NOs: 44-48, 52, and 53, which are provided below. Another exemplary codon optimized nucleic acid sequence encoding a Cas9 molecule of S. aureus comprises the nucleotides 1293-4451 of SEQ ID NO: 55. An amino acid sequence of an S. aureus Cas9 molecule is set forth in SEQ ID NO: 49. An amino acid sequence of a Streptococcus pyogenes Cas9 (with D10A, H849A mutations) is set forth in SEQ ID NO: 54.

b. Fusion Protein

Alternatively or additionally, the CRISPR/Cas-based gene editing system can include a fusion protein. The fusion protein can comprise two heterologous polypeptide domains, wherein the first polypeptide domain comprises a DNA binding protein such as a Cas protein, a zinc finger protein, or a TALE protein, and the second polypeptide domain has an activity such as transcription activation activity, transcription repression activity, transcription release factor activity, histone modification activity, nuclease activity, nucleic acid association activity, methylase activity, or demethylase activity. The fusion protein can include a first polypeptide domain such as a Cas9 protein or a mutated Cas9 protein, fused to a second polypeptide domain that has an activity such as transcription activation activity, transcription repression activity, transcription release factor activity, histone modification activity, nuclease activity, nucleic acid association activity, methylase activity, or demethylase activity. In some embodiments, the second polypeptide domain has transcription activation activity. In some embodiments, the second polypeptide domain comprises a synthetic transcription factor. The fusion protein may include one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

i) Transcription Activation Activity

The second polypeptide domain can have transcription activation activity, i.e., a transactivation domain. For example, gene expression of endogenous mammalian genes, such as human genes, can be achieved by targeting a fusion protein of a first polypeptide domain, such as dCas9 or dCas12, and a transactivation domain to mammalian promoters via combinations of gRNAs. The transactivation domain can include a VP 16 protein, multiple VP 16 proteins, such as a VP48 domain or VP64 domain, p65 domain of NF kappa B transcription activator activity, or p300. For example, the fusion protein may be dCas9-VP64. In other embodiments, the Cas9 protein may be VP64-dCas9-VP64 (SEQ ID NO: 57, encoded by SEQ ID NO: 58). In other embodiments, the fusion protein that activates transcription may be dCas9-p300. In some embodiments, p300 may comprise a polypeptide of SEQ ID NO: 59 or SEQ ID NO: 60.

ii) Transcription Repression Activity

The second polypeptide domain can have transcription repression activity. The second polypeptide domain can have a Kruppel associated box activity, such as a KRAB domain, ERF repressor domain activity, Mxil repressor domain activity, SID4X repressor domain activity, Mad-SID repressor domain activity, or TATA box binding protein activity. For example, the fusion protein may be dCas9-KRAB.

iii) Transcription Release Factor Activity

The second polypeptide domain can have transcription release factor activity.

The second polypeptide domain can have eukaryotic release factor 1 (ERF1) activity or eukaryotic release factor 3 (ERF3) activity.

iv) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 may comprise a polypeptide of SEQ ID NO: 59 or SEQ ID NO: 60.

v) Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease.

vi) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, TAL effector DNA-binding domain.

vii) Methylase Activity

The second polypeptide domain can have methylase activity, which involves transferring a methyl group to DNA, RNA, protein, small molecule, cytosine or adenine. In some embodiments, the second polypeptide domain includes a DNA methyltransferase.

viii) Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Teti.

c. gRNA

The CRISPR/Cas-based gene editing system includes at least one gRNA molecule. For example, the CRISPR/Cas-based gene editing system may include two gRNA molecules. The gRNA provides the targeting of a CRISPR/Cas-based gene editing system. The gRNA is a fusion of two noncoding RNAs: a crRNA and a tracrRNA. In some embodiments, the polynucleotide includes a crRNA, and/or a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to cleave the target nucleic acid. The “target region,” “target sequence,” or “protospacer,” refers to the region of the target gene (e.g., a Pax7 gene) to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The scaffold may comprise a polynucleotide sequence of SEQ ID NO: 85. The CRISPR/Cas9-based gene editing system may include at least one gRNA, wherein the gRNAs target different DNA sequences. The target DNA sequences may be overlapping. The target sequence or protospacer is followed by a PAM sequence at the 3′ end of the protospacer in the genome. Different Type II systems have differing PAM requirements. For example, the Streptococcus pyogenes Type II system uses an “NGG” sequence, where “N” can be any nucleotide. In some embodiments, the PAM sequence may be ‘NGG’, where ‘N’ can be any nucleotide. In some embodiments, the PAM sequence may be NNGRRT (SEQ ID NO: 40) or NNGRRV (SEQ ID NO: 41).

The number of gRNA molecule encoded by a genetic construct (e.g., an AAV vector) can be at least 1 gRNA, at least 2 different gRNA, at least 3 different gRNA at least 4 different gRNA, at least 5 different gRNA, at least 6 different gRNA, at least 7 different gRNA, at least 8 different gRNA, at least 9 different gRNA, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNAs encoded by a presently disclosed vector can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs. In certain embodiments, the genetic construct (e.g., an AAV vector) encodes one gRNA molecule, i.e., a first gRNA molecule, and optionally a Cas9 molecule. In certain embodiments, a first genetic construct (e.g., a first AAV vector) encodes one gRNA molecule, i.e., a first gRNA molecule, and optionally a Cas9 molecule, and a second genetic construct (e.g., a second AAV vector) encodes one gRNA molecule, i.e., a second gRNA molecule, and optionally a Cas9 molecule.

The gRNA molecule comprises a targeting domain, which is a polynucleotide sequence complementary to the target DNA sequence followed by a PAM sequence. The gRNA may comprise a “G” at the 5′ end of the targeting domain or complementary polynucleotide sequence. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The gRNA may target a region within or near the Pax7 gene, or within or near a regulatory element or promoter of the Pax7 gene. In certain embodiments, the gRNA can target at least one of exons, introns, the promoter region, the enhancer region, or the transcribed region of the gene. The gRNA may target Pax7 or a promoter or regulatory element of the Pax7 gene. In some embodiments, the gRNA targets a Pax7 promoter. The gRNA may include a targeting domain that comprises a polynucleotide sequence corresponding to at least one of SEQ ID NOs: 1-8 or 69-76 or 77-84, or a complement thereof or a variant thereof, as shown in TABLE 1. In some embodiments, the gRNA targets a polynucleotide sequence comprising the complement of at least one of SEQ ID NOs: 1-8. In some embodiments, the gRNA is encoded by a polynucleotide sequence comprising at least one of SEQ ID NOs: 1-8. In some embodiments, the gRNA comprises a polynucleotide sequence selected from SEQ ID NOs: 69-76. In some embodiments, the gRNA binds and targets a polynucleotide comprising a sequence selected from SEQ ID NOs: 77-84, respectively, in TABLE 4.

TABLE 1
gRNAs that activate endogenous Pax7.

| SEQ | | SEQ | |
|---|---|---|---|
| ID | | ID | |
| NO | gRNA seguence | NO | gRNA |
| 1 | GGCCGGGGACTCGGCGGATC | 69 | GGCCGGGGACUCGGCGGAUC |
| 2 | TCCCCGGCTCGACCTCGTTT | 70 | UCCCCGGCUCGACCUCGUUU |
| 3 | CCAGGGCGCAAGGGAGCGG | 71 | CCAGGGCGCAAGGGAGCGG |
| 4 | TCCTCCGCTCCCTTGCGCCC | 72 | UCCUCCGCUCCCUUGCGCCC |
| 5 | GGGGGCGCGAGTGATCAGCT | 73 | GGGGGCGCGAGUGAUCAGCU |
| 6 | CGGGTTTCAGGGCTGGACGG | 74 | CGGGUUUCAGGGCUGGACGG |
| 7 | TGGTCCGGAGAAAGAAGGCG | 75 | UGGUCCGGAGAAAGAAGGCG |
| 8 | AGCGCCAGAGCGCGAGAGCG | 76 | AGCGCCAGAGCGCGAGAGCG |

TABLE 4
Target seguences of the gRNAs that
activate endogenous Pax7

| SEQ ID NO | gRNA target seguence |
|---|---|
| 77 | GATCCGCCGAGTCCCCGGCC |
| 78 | AAACGAGGTCGAGCCGGGGA |
| 79 | CCGCTCCCTTGCGCCCTGG |
| 80 | GGGCGCAAGGGAGCGGAGGA |
| 81 | AGCTGATCACTCGCGCCCCC |
| 82 | CCGTCCAGCCCTGAAACCCG |
| 83 | CGCCTTCTTTCTCCGGACCA |
| 84 | CGCTCTCGCGCTCTGGCGCT |

Single or multiplexed gRNAs can be designed to activate expression of Pax7, thereby differentiating a stem cell into a skeletal muscle progenitor cell. Following treatment with a construct or system as detailed herein, a stem cell may be differentiated into a skeletal muscle progenitor cell. Genetically corrected stem or patient cells may be transplanted into a subject.

d. DNA Targeting System

Further provided herein are DNA targeting systems or compositions that comprise such genetic constructs. The DNA targeting compositions include at least one gRNA molecule (e.g., two gRNA molecules) that targets a gene, as described above. The at least one gRNA molecule can bind and recognize a target region.

In some embodiments, the DNA targeting composition includes a first gRNA and a second gRNA. In some embodiments, the first gRNA molecule and the second gRNA molecule comprise different targeting domains.

The DNA targeting composition may further include at least one Cas molecule or a fusion protein. In some embodiments as detailed above, the DNA targeting composition further includes at least one dCas9 protein or fusion protein. In some embodiments, the Cas9 molecule or fusion protein recognizes a PAM of either NNGRRT (SEQ ID NO: 40) or NNGRRV (SEQ ID NO: 41). In some embodiments, the DNA targeting composition includes a nucleotide sequence set forth in SEQ ID NO: 55. In certain embodiments, the vector is configured to form a first and a second double strand break in a segment within or near the Pax7 gene.

The DNA targeting composition may further comprise a donor DNA or a transgene.

### 4. Genetic Constructs

The DNA targeting system, or one or more components thereof, may be encoded by or comprised within a genetic construct. Genetic constructs may include polynucleotides such as vectors and plasmids. The construct may be recombinant. In some embodiments, the genetic construct comprises a promoter that is operably linked to the polynucleotide encoding at least one gRNA molecule and/or a Cas molecule or fusion protein. In some embodiments, the genetic construct comprises a promoter that is operably linked to the polynucleotide encoding at least one gRNA molecule and/or a dCas molecule or fusion protein. In some embodiments, the genetic construct comprises a promoter that is operably linked to the polynucleotide encoding at least one gRNA molecule and/or a Cas9 molecule or fusion protein. In some embodiments, the promoter is operably linked to the polynucleotide encoding a first gRNA molecule, a second gRNA molecule, and/or a Cas9 molecule or fusion protein. The genetic construct may be present in the cell as a functioning extrachromosomal molecule. The genetic construct may be a linear minichromosome including centromere, telomeres, or plasmids or cosmids. The genetic construct may be transformed or transduced into a cell. The genetic construct may be formulated into any suitable type of delivery vehicle including, for example, a viral vector, lentiviral expression, mRNA electroporation, and lipid-mediated transfection. Further provided herein is a cell transformed or transduced with a DNA targeting system or component thereof as detailed herein. The cell may be, for example, a stem cell, or a fibroblast. In some embodiments, the stem cell is a pluripotent stem cells. In some embodiments, the fibroblast is a skin fibroblast.

Further provided herein is a viral delivery system. In some embodiments, the vector is an adeno-associated virus (AAV) vector. The AAV vector is a small virus belonging to the genus Dependovirus of the Parvoviridae family that infects humans and some other primate species. AAV vectors may be used to deliver CRISPR/Cas9-based gene editing systems using various construct configurations. For example, AAV vectors may deliver Cas9 and gRNA expression cassettes on separate vectors or on the same vector. Alternatively, if the small Cas9 proteins, derived from species such as Staphylococcus aureus or Neisseria meningitidis, are used then both the Cas9 and up to two gRNA expression cassettes may be combined in a single AAV vector within the 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-846). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

### 5. Pharmaceutical Compositions

Further provided herein are pharmaceutical compositions comprising the above-described genetic constructs or DNA targeting systems. The DNA targeting systems, or at least one component thereof, as detailed herein may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent may be a polyanion, polycation, including poly-L-glutamate (LGS), or lipid. The transfection facilitating agent is poly-L-glutamate, and more preferably, the poly-L-glutamate is present in the composition for genome editing in skeletal muscle or cardiac muscle at a concentration less than 6 mg/mL. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the composition may also include a transfection facilitating agent such as lipids, liposomes, including lecithin liposomes or other liposomes known in the art, as a DNA-liposome mixture (see for example International Patent Publication No. WO9324840), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid.

### 6. Administration

The DNA targeting systems, or at least one component thereof, as detailed herein, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed DNA targeting systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the DNA targeting system, genetic construct, or composition comprising the same, is administered to a subject intramuscularly, intravenously, or a combination thereof. For veterinary use, the DNA targeting systems, genetic constructs, or compositions comprising the same may be administered as a suitably acceptable formulation in accordance with normal veterinary practice. The veterinarian may readily determine the dosing regimen and route of administration that is most appropriate for a particular animal. The DNA targeting systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound.

The DNA targeting systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the skeletal muscle or cardiac muscle. For example, the composition may be injected into the tibialis anterior muscle or tail.

In some embodiments, the DNA targeting system, genetic construct, or composition comprising the same, is administered by 1) tail vein injections (systemic) into adult mice; 2) intramuscular injections, for example, local injection into a muscle such as the TA or gastrocnemius in adult mice; 3) intraperitoneal injections into P2 mice; or 4) facial vein injection (systemic) into P2 mice. In some embodiments, the DNA targeting system, genetic construct, or composition comprising the same, is administered to a human by intravenous or intramuscular injection.

Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein. In some embodiments, the Cas9 is a dCas9 or fusion protein.

Any of the delivery methods and/or routes of administration detailed herein can be utilized with a myriad of cell types, for example, those cell types currently under investigation for cell-based therapies, including, but not limited to, immortalized myoblast cells, such as wild-type and patient derived lines, primal dermal fibroblasts, stem cells such as induced pluripotent stem cells, bone marrow-derived progenitors, skeletal muscle progenitors, human skeletal myoblasts from patients, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells. The stem cell may be a human pluripotent stem cell. The stem cell may be an induced pluripotent stem cell (iPSC). The stem cell may be an embryonic stem cell (ESC).

### 7. Methods

a. Methods of Activating Endogenous Myogenic Transcription Factor Pax7

Provided herein are methods for activating endogenous myogenic transcription factor Pax7 in a cell. The method may include administering to the cell a DNA targeting system as detailed herein, an isolated polynucleotide sequence as detailed herein, a vector as detailed herein, a cell as detailed herein, or a combination thereof. In some embodiments, endogenous expression of Pax7 mRNA is increased in the skeletal muscle progenitor cell. In some embodiments, expression of Myf5, MyoD, MyoG, or a combination thereof, is increased in the skeletal muscle progenitor cell. In some embodiments, the stem cell is induced into myogenic differentiation. In some embodiments, the skeletal muscle progenitor cell maintains Pax7 expression after at least about 2, at least about 3, at least about 4, at least about 5, at least about 6, at least about 7, at least about 8, at least about 9, at least about 10, at least about 11, at least about 12, at least about 13, at least about 14, or at least about 15 passages.

b. Methods of Differentiating a Stem Cell into a Skeletal Muscle Progenitor Cell

Provided herein are methods of differentiating a stem cell into a skeletal muscle progenitor cell. The method may include administering to the cell a DNA targeting system as detailed herein, an isolated polynucleotide sequence as detailed herein, a vector as detailed herein, a cell as detailed herein, or a combination thereof. In some embodiments, endogenous expression of Pax7 mRNA is increased in the skeletal muscle progenitor cell. In some embodiments, expression of Myf5, MyoD, MyoG, or a combination thereof, is increased in the skeletal muscle progenitor cell. In some embodiments, the stem cell is induced into myogenic differentiation. In some embodiments, the skeletal muscle progenitor cell maintains Pax7 expression after at least about 2, at least about 3, at least about 4, at least about 5, at least about 6, at least about 7, at least about 8, at least about 9, at least about 10, at least about 11, at least about 12, at least about 13, at least about 14, or at least about 15 passages.

c. Methods of Treating a Subject

Provided herein are methods for activating endogenous myogenic transcription factor Pax7 in a cell. The method may include administering to the cell a DNA targeting system as detailed herein, an isolated polynucleotide sequence as detailed herein, a vector as detailed herein, a cell as detailed herein, or a combination thereof. In some embodiments, endogenous expression of Pax7 mRNA is increased in the subject. In some embodiments, expression of Myf5, MyoD, MyoG, or a combination thereof, is increased in the subject. In some embodiments, a cell in the subject is induced into myogenic differentiation. In some embodiments, the level of dystrophin+ fibers in the subject is increased. In some embodiments, muscle regeneration in the subject is increased.

### 8. Examples

### Example 1

### Materials and Methods

gRNA design, transfection, and plasmid construction. Pax7 promoter targeting gRNAs were designed using crispr.mit.edu and cloned into a gRNA vector (Addgene plasmid 41824). Candidate Pax7 gRNAs were transiently transfected with Lipofectamine 3000 on the second day of CHIRON99021-induced differentiation of H9 ESCs constitutively expressing VP64-dCas9-VP64. Cells were harvested after 6 days for qRT-PCR analysis of Pax7. For doxycycline (dox)-inducible expression of VP64-dCas9-VP64, the pLV-hUBC-VP64dCas9VP64-T2A-GFP plasmid (Addgene plasmid 59791) served as the source vector for generating the pLV-tightTRE-VP64dCas9VP64-T2A-mCherry. The Pax7 gRNA was cloned into a pLV-hU6-gRNA-PGK-rtTA3-Blast that was generated using pLV-CMV-rtTA3-Blast as the source vector (Addgene plasmid 26429). The Pax7 cDNA (DNASU plasmid HsCD00443491) was cloned into a lentiviral construct to generate pLV-tightTRE-Pax7-P2A-mCherry construct. The PAX7-A sequence was confirmed to be the same as the PAX7 sequence used in previous directed differentiation papers. The PAX7-B sequence was obtained by PCR of mRNA isolated from cells treated with VP64dCas9VP64+gRNA and cloned into a lentiviral tightTRE-PAX7-B-P2A-mCherry construct. Sequences of the target sequences of the gRNAs are shown in TABLE 2. Primers used are shown in TABLE 3.

TABLE 2

| gRNA | SEQ | Protospacer Seguence | Position Relative |
|---|---|---|---|
| # | ID # | (5′-3′) | to TSS |
| 1 | 1 | GGCCGGGGACTCGGCGGATC | −490 |
| 2 | 2 | TCCCCGGCTCGACCTCGTTT | −351 |
| 3 | 3 | CCAGGGCGCAAGGGAGCGG | −278 |
| 4 | 4 | TCCTCCGCTCCCTTGCGCCC | −282 |
| 5 | 5 | GGGGGCGCGAGTGATCAGCT | −137 |
| 6 | 6 | CGGGTTTCAGGGCTGGACGG | −70 |
| 7 | 7 | TGGTCCGGAGAAAGAAGGCG | +30 |
| 8 | 8 | AGCGCCAGAGCGCGAGAGCG | +158 |

TABLE 3

| | | | Cycling |
|---|---|---|---|
| Target | Forward Primer (5′-3′) | Reverse Primer (5′-3′) | Condition |
| GAPDH | GAAGGTGAAGGTCGGAGTC | GAAGATGGTGATGGGATTTC | 95° C. 5 s |
| | (SEQ ID NO: 9) | (SEQ ID NO: 10) | 58° C. |
| | | | 20 s × 40 |
| PAX7 | CAGCAAGCCCAGACAGGTGG | GCACGCGGCTAATCGAACTC | 95° C. 5 s |
| | (SEQ ID NO: 11) | (SEQ ID NO: 12) | 58° C. |
| | | | 20 s × 40 |
| MYF5 | AATTTGGGGACGAGTTTGTG | CATGGTGGTGGACTTCCTCT | 95° C. 5 s |
| | (SEQ ID NO: 13) | (SEQ ID NO: 14) | 58° C. |
| | | | 20 s × 40 |
| MYOD | AGACTGCCAGCACTTTGCTA | GTAGCTCCATATCCTGGCGG | 95° C. 5 s |
| | (SEQ ID NO: 15) | (SEQ ID NO: 16) | 58° C. |
| | | | 20 s × 40 |
| MYOG | GGTGCCCAGCGAATGC (SEQ | TGATGCTGTCCACGATGGA | 95° C. 5 s |
| | ID NO: 17) | (SEQ ID NO: 18) | 58° C. |
| | | | 20 s × 40 |
| Endogenous | GCTACAAGGTGGTGTCAGGG | GAGCCATAGTACGGAAGCAGAG | 95° C. 5 s |
| PAX7 | T (SEQ ID NO: 19) | (SEQ ID NO: 20) | 58° C. |
| Isoform 1/2 | | | 20 s × 40 |
| (PAX7-A) | | | |
| Endogenous | TCTGGCCAAAAATGTGAGCC | GGGTCAGTTAGGGTTGGGC | 95° C. 5 s |
| PAX7 | T (SEQ ID NO: 21) | (SEQ ID NO: 22) | 58° C. |
| Isoform 3 | | | 20 s × 40 |
| (PAX-7B) | | | |
| T | TGCTTCCCTGAGACCCAGTT | GATCACTTCTTTCCTTTGCATCAA | 95° C. 5 s |
| | (SEQ ID NO: 23) | G | 58° C. |
| | | (SEQ ID NO: 24) | 20 s × 40 |
| TBX6 | CAACCCCGCATACACCTAGT | CGTCTCGCTCCCTCTTACAG | 95° C. 5s |
| | (SEQ ID NO: 25) | (SEQ ID NO: 26) | 58° C. |
| | | | 20 s × 40 |
| MSGN1 | AACCTGCGCGAGACTTTCC | ACAGCTGGACAGGGAGAAGA | 95° C. 5 s |
| | (SEQ ID NO: 27) | (SEQ ID NO: 28) | 58° C. |
| | | | 20 s × 40 |
| Pax3 | CTCACCTCAGGTAATGGGAC | CGTGGTGGTAGGTTCCAGAC | 95° C. 5 s |
| | T (SEQ ID NO: 29) | (SEQ ID NO: 30) | 58° C. |
| | | | 20 s × 40 |
| PAX7 ChIP | CGGGGCTCTGACATTACACA | GCCAGAGTCCGCCCTATTTC | 95° C. 5 s |
| 1, −731 bp | (SEQ ID NO: 61) | (SEQ ID NO: 62 | 60° C. |
| | | | 20 s × 40 |
| PAX7 ChIP | TATTGGTCCTCCGCTCCCTT | GTGAGCGCGATCTGATAGGT | 95° C. 5 s |
| 2, −289 bp | (SEQ ID NO: 63) | (SEQ. ID NO: 64) | 60° C. |
| | | | 20 s × 40 |
| PAX7 ChIP | TTGCCGACTTTGGATTCGTC | TCCAAAGGGAATCCCGTGC | 95° C. 5 s |
| 3, +562 bp | (SEQ ID NO: 65) | (SEQ ID NO: 66) | 60° C. |
| | | | 20 s × 40 |
| PAX7 ChIP | CGCAGGGCTGAAATTCTGGT | AGAGCCGAGAAACTGTCAGG | 95° C. 5 s |
| 4, +926 | (SEQ ID NO: 67) | (SEQ ID NO: 68) | 60° C. |
| | | | 20 s × 40 |

Lentiviral production. HEK293T cells were obtained from the American Tissue Collection Center (ATCC) and purchased through the Duke University Cancer Center Facilities and were cultured in Dulbecco's Modified Eagle's Medium (Invitrogen) supplemented with 10% FBS (Sigma) and 1% penicillin/streptomycin (Invitrogen) at 37° C. with 5% CO2. Approximately 3.5 million cells were plated per 10 cm TCPS dish. Twenty-four hours later, the cells were transfected using the calcium phosphate precipitation method with pMD2.G (Addgene #12259) and psPAX2 (Addgene #12260) second generation envelope and packaging plasmids. The medium was exchanged 12 hours post-transfection, and the viral supernatant was harvested 24 and 48 hours after this medium change. The viral supernatant was pooled and centrifuged at 500 g for 5 minutes, passed through a 0.45 μm filter, and concentrated to 20× using Lenti-X Concentrator (Clontech) in accordance with the manufacturer's protocol. Undifferentiated hPSCs were transduced with the pLV-hU6-gRNA-PGK-rtTA3-Blast and cells were selected with 2 μg/mL of blasticidin (Thermo) to generate homogenous population of stably transduced cells. Just prior to differentiation, hPSCs were resuspended and plated with lentivirus encoding inducible VP64-dCas9-VP64 or Pax7 cDNA.

Cell culture. H9 ESCs (obtained from the WiCell Stem Cell Bank) and DU11 iPSCs were used for these studies. DU11 iPSCs were generated by the Duke iPSC Shared Resource Facility via episomal reprogramming of BJ fibroblasts from a healthy male newborn (ATCC cell line, CRL-2522). Stable and correct karyotype and pluripotency of the cells was confirmed. hPSCs were maintained in mTeSR (Stem Cell Technologies) and plated on tissue culture treated plates coated with ES-qualified matrigel (Corning). For differentiation, hPSCs were dissociated into single cells with Accutase (Stem Cell Technologies) and plated on matrigel coated plates at 2.3-3.3×10⁴/cm² in mTeSR medium supplemented with 10 μM Y27632 (Stem Cell Technologies). The following day, mTeSR medium was replaced with E6 media supplemented with 10 μM CHIR99021 (Sigma) to initiate mesoderm differentiation. After 2 days, CHIR99021 was removed and cells were maintained in E6 media with 10 ng/mL FGF2 (Sigma) and 1 μg/mL of doxycycline (dox) (Sigma).

Fluorescence activated cell sorting and expansion of sorted cells. At day 14 after induction of differentiation, cells were dissociated with 0.25% Trypsin-EDTA (Thermo) and washed with neutralizing media (10% FBS in DMEM/F12). Cells were pelleted by centrifugation and resuspended in flow media (5% FBS in PBS). Cells were sorted for mCherry expression, pelleted, resuspended in growth media (E6 supplemented with 10 ng/mL FGF2 and 1 μg/mL dox) and plated on matrigel-coated plates. Cells were passaged every 3-4 days at ˜80% confluency. Terminal differentiation was induced by withdrawing dox from the medium in 100% confluent cultures.

Flow cytometry analysis. For flow cytometry analysis of surface markers, cells were harvested during the proliferation phase at day 20 of differentiation. Cells were dissociated with 0.25% Trypsin-EDTA, washed with PBS, then resuspended in flow buffer (PBS with 5% FBS). Cells were incubated with the following conjugated antibodies at 0.25 μg/10⁶ cells: IgG1-K isotype control-FITC (eBioscience 11-4714-41), CD56-FITC (eBioscience 11-0566-41), or CD29-FITC (eBioscience 11-0299-41). Cells were analyzed on SONY SH800 flow cytometer.

Cell transplantation into Immunodeficient mice. All animal experiments were conducted under protocols approved by the Duke Institutional Animal Care and Use Committee. 7 week old female NOD.SCID.gamma mice (Duke CCIF Breeding Core) were used for these in vivo studies. Prior to intramuscular cell transplantation, mice were pre-injured with 30 μL of 1.2% BaCl2 (Sigma). 24 hours later, MPCs from differentiated iPSCs or ESCs were injected into the tibialis anterior (TA) muscle (5×10⁵ cells/15 μL Hank's Balanced Salt Solution). Four weeks after injection, mice were euthanized and the TA muscles were harvested.

Immunofluorescence staining of cultured cells and tissue sections. Cultured cells were plated on autoclaved glass coverslips (1 mm, Thermo) coated with matrigel for immunofluorescence staining during the proliferation phase. For differentiation, cells were grown to confluency and differentiated on 24 well tissue culture plates coated with matrigel, and immunofluorescence staining was performed directly in the well. Cells were fixed with 4% PFA for 15 min and permeabilized in blocking buffer (PBS supplemented with 3% BSA and 0.2% Triton X-100) for 1 hr at room temperature. Samples were incubated overnight at 4° C. with the following antibodies: Pax7 (1:20, Developmental Studies Hybridoma Bank), Myosin Heavy Chain MF20 (1:200, DSHB), Myf5 (1:200, Santa Cruz sc-302) and MyoD 5.8A (1:200, Santa Cruz sc-32758). Samples were washed with PBS for 15 min and incubated with compatible secondary antibodies diluted 1:500 from Invitrogen and DAPI for 1 hr at room temperature. Samples were washed for 15 min with PBS and coverslips were mounted with ProLong Gold Antifade Reagent (Invitrogen) or wells were kept in PBS and imaged using conventional fluorescence microscopy. Harvested TA muscles were mounted and frozen in Optimal Cutting Temperature (OCT) compound cooled in liquid nitrogen. Serial 10 μm cryosections were collected. Cryosections were fixed with 2% PFA for 5 min and permeabilized with PBS+0.2% Triton-X for 10 minutes. Blocking buffer (PBS supplemented with 5% goat serum, 2% BSA, and 0.1% Triton X-100) was applied for 1 hr at room temperature. Samples were incubated overnight at 4° C. with a combination of the following antibodies: human-specific MANDYS106 (1:200, Sigma MABT827), human-specific Lamin A/C (1:100, Thermo MA31000), Pax7 (1:10, Developmental Studies Hybridoma Bank), or Laminin (1:200, Sigma L9393). Samples were washed with PBS for 15 min and incubated with compatible secondary antibodies diluted 1:500 from Invitrogen and DAPI for 1 hr at room temperature. Samples were washed for 15 min with PBS and slides were mounted with ProLong Gold Antifade Reagent (Invitrogen) and imaged using conventional fluorescence microscopy.

Quantitative Reverse Transcription PCR. RNA was isolated using the RNeasy Plus RNA isolation kit (Qiagen). cDNA was synthesized with the SuperScript VILO cDNA Synthesis Kit (Invitrogen). Real-time PCR using PerfeCTa SYBR Green FastMix (Quanta Biosciences) was performed with the CFX96 Real-Time PCR Detection System (Bio-Rad). The results are expressed as fold-increase expression of the gene of interest normalized to GAPDH expression using the ΔΔCt method.

Chromatin Immunoprecipitation (ChIP) qPCR. ChIP was performed using the EpiQuik ChIP Kit (EpiGentek) according to manufacturer's instructions. Soluble chromatin was immunoprecipitated with antibodies against H3K27ac and H3K4me3 (abcam), and gDNA was purified for qPCR analysis. All sequences for ChIP-qPCR primers can be found in TABLE 3. qPCR was performed using PerfeCTa SYBR Green FastMix (Quanta BioSciences), and the data are presented as fold change gDNA relative to negative control (gRNA only) and normalized to a region of the GAPDH locus.

RNA-Seq. RNA was extracted from freshly sorted cells at day 14 of differentiation using the Total RNA Purification Plus Micro Kit (Norgen). Library preparation and sequencing was performed by GENEWIZ on an Illumina HiSeq in the 2×150 bp sequencing configuration. All RNA-seq samples were first validated for consistent quality using FastQC v0.11.2 (Babraham Institute). Raw reads were trimmed to remove adapters and bases with average quality score (Q) (Phred33) of <20 using a 4 bp sliding window (SLIDINGWINDOW:4:20) with Trimmomatic v0.32 (Bolger et al. Bioinformatics 2014, 30, 2114-2120). Trimmed reads were subsequently aligned to the primary assembly of the GRCh38 human genome using STAR v2.4.1a (Dobin et al. Bioinformatics 2013, 29, 15-21) removing alignments containing non-canonical splice junctions (--outFilterIntronMotifs RemoveNoncanonical). Aligned reads were assigned to genes in the GENCODE v19 comprehensive gene annotation (Harrow et al. Genome Res. 2012, 22, 1760-1774) using the featureCounts command in the subread package with default settings (v1.4.6-p4) (Liao et al. Nucleic Acids Res. 2013, 41, e108-e108). The subsequent counts were normalized for each replicate using the R package DESeq2 after filtering out genes that were not sufficiently quantified, and normalized values were used for analysis. Heatmaps were generated using the pheatmap package in R software. Biological processes and pathways were generated using Enrichr (Chen et al. BMC Bioinformatics 2013, 14, 128), a web-based online tool. For estimating transcript and gene abundances, Transcript Per Million (TPMs) were computed using the rsem-calculate-expression function in the RSEM v1.2.21 package (Li and Dewey. BMC Bioinformatics 2011, 12, 323).

### Example 2

### Developing Conditions for VP64-dCas9-VP64-Mediated Endogenous Pax7 Activation in hPSCs

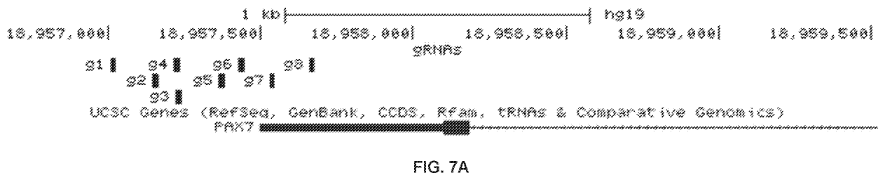

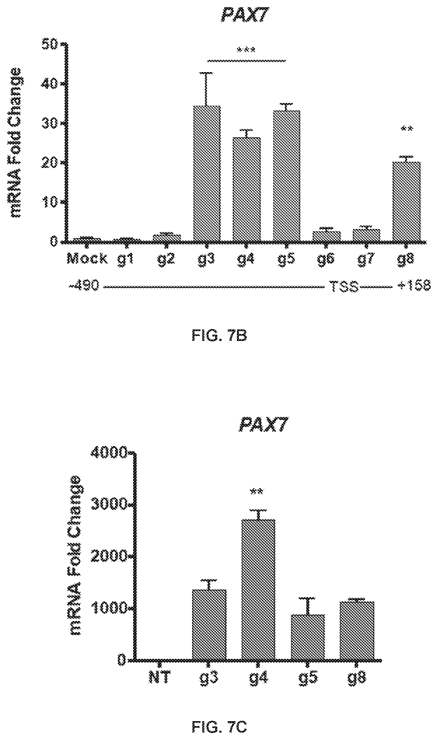

During embryonic differentiation, PAX7 and its paralog PAX3 specify myogenic cells within the paraxial mesoderm. Differentiation of hPSCs into paraxial mesoderm cells can be initiated by CHIR99021, a GSK3 inhibitor (Tan et al. Stem Cells Dev. 2013, 22, 1893-1906). Two human pluripotent stem cell lines, H9 ESCs and DU11 iPSCs, were used for differentiation studies. For targeted gene activation, we used the dCas9 with the VP64 domain fused to both the N- and C-termini (VP64-dCas9-VP64), which we previously showed to be ˜10-fold more potent than a single VP64 fusion. To test the efficacy of VP64-dCas9-VP64-mediated activation of PAX7, we designed 8 gRNAs spanning −490 to +158 base pairs relative to the transcription start site of the human PAX7 gene (FIG. 7A). H9 ESCs stably expressing VP64-dCas9-VP64 were differentiated into paraxial mesoderm cells with addition of CHIR99021 in E6 medium for 2 days, as previously described (Shelton et al. Stem Cell Rep. 2014, 3, 516-529). Cells were transfected with the individual gRNAs and samples were harvested 6 days later for gene expression analysis using qRT-PCR. 4 out of the 8 gRNAs significantly upregulated PAX7 compared to mock transfected cells (FIG. 7B). In a second screen, we packaged the 4 individual gRNAs that performed best in the transfection experiment into lentiviruses to achieve more stable and robust expression. Cells were harvested at 8 days post-transduction. gRNA #4 was identified as the most potent gRNA and was used for subsequent studies (FIG. 7C).

### Example 3

### VP64-dCas9-VP64-Mediated Differentiation of hPSCs into Myogenic Progenitor Cells

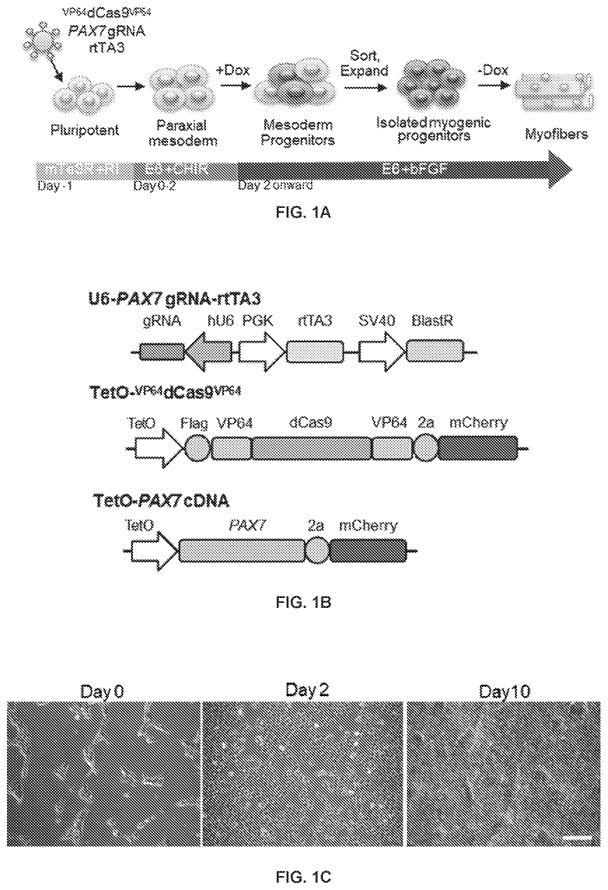

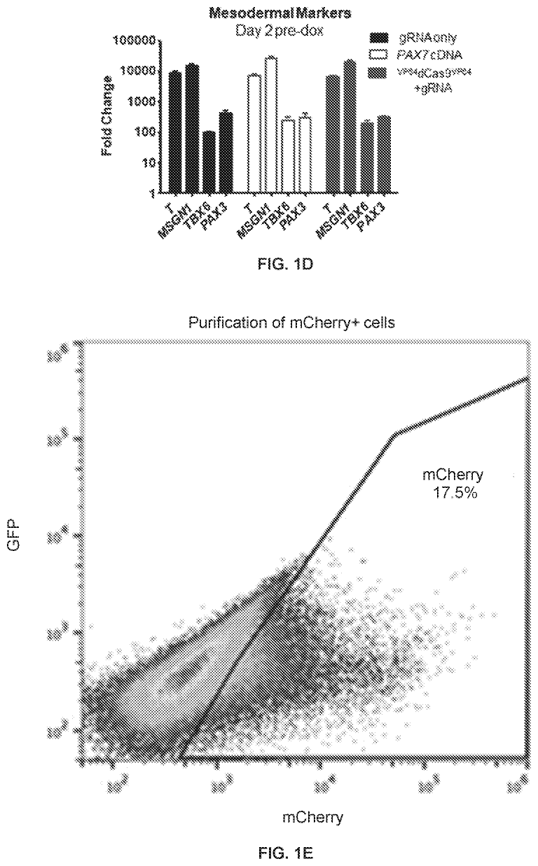

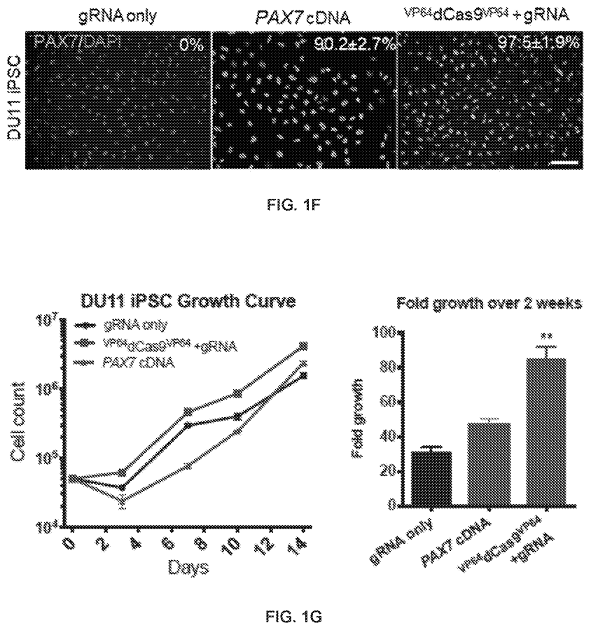

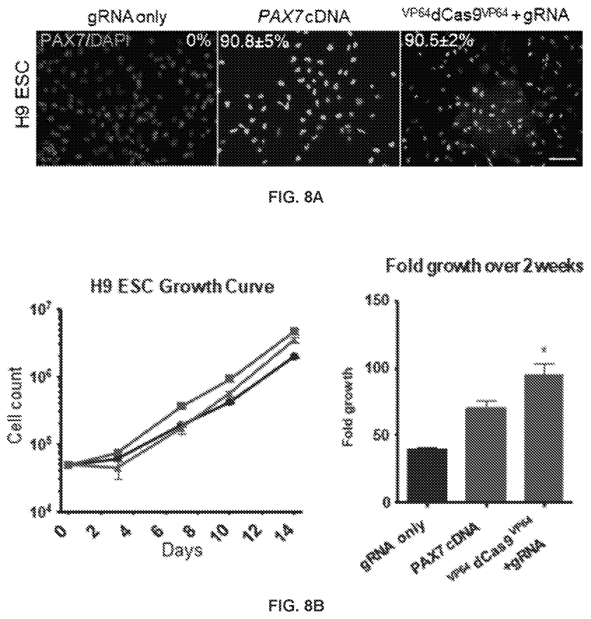

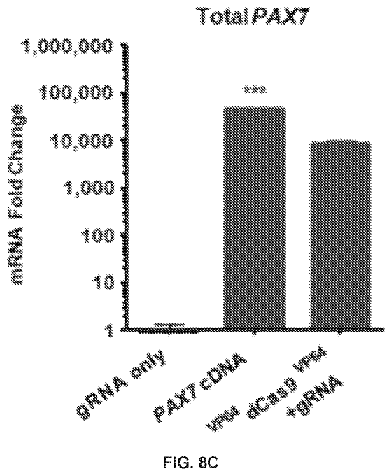

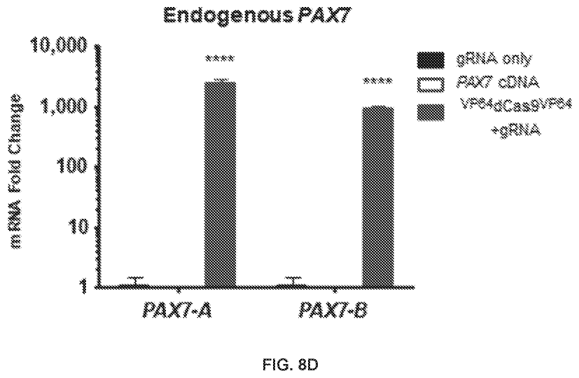

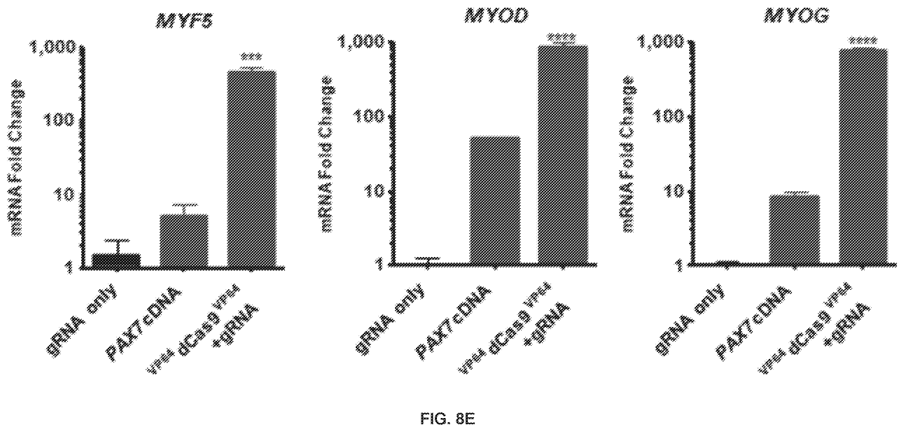

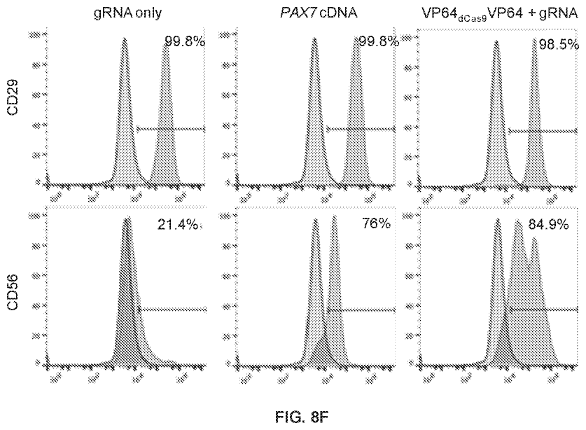

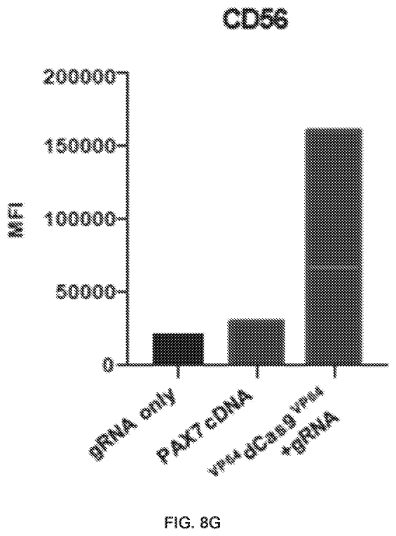

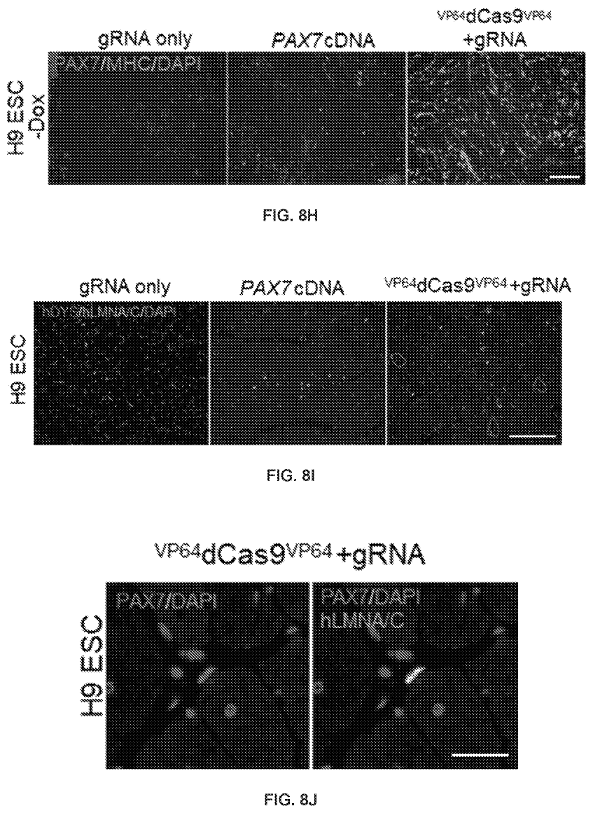

Next, we tested the hypothesis that endogenous PAX7 activation in paraxial mesoderm cells would be sufficient for generating myogenic progenitor cells (MPCs) with the potential to differentiate into myotubes in vitro (FIG. 1A). Prior to differentiation, hPSCs were transduced with a lentivirus expressing the PAX7 promoter-targeting gRNA, a reverse tetracycline transactivator (rtTA), and a blasticidin resistance gene. Cells were selected with blasticidin for stable expression of the vector and then transduced with an additional lentivirus encoding either doxycycline (dox)-inducible VP64-dCas9-VP64 or the PAX7 cDNA, which also included a co-transcribed mCherry reporter gene (FIG. 1B). hPSCs were differentiated with CHIR99021 for 2 days and then maintained in E6 medium with dox and FGF2 to support MPC proliferation (FIG. 1C) (Pawlikowski et al. Dev. Dyn. 2017, 246, 359-367). Addition of CHIR99021 induced paraxial mesodermal differentiation, as indicated by high levels of pan-mesoderm marker Brachyury (7), paraxial mesoderm markers MSGN1 and TBX6, and premyogenic mesoderm marker PAX3 at the mRNA level (FIG. 1D). Transduced cells were sorted based on mCherry expression after two weeks of growth (FIG. 1E). mCherry+ cells accounted for ˜20% of cells transduced with VP64-dCas9-VP64 compared to ˜50% with PAX7 cDNA transduced cells. This is likely due to the larger size of VP64-dCas9-VP64 vector compared to the PAX7 cDNA vector (7.9 kb between LTRs vs. 4.9 kb) resulting in reduced lentiviral titers. These purified MPCs were maintained in serum-free E6 medium supplemented with dox and FGF2 and passaged when cells reached ˜80% confluency. Sorted cells demonstrated high purity of PAX7+ cells in both the endogenous-activated cells and exogenous cDNA-expressing cells when protein expression was assessed by immunofluorescence staining 5 days after sorting (FIG. 1F and FIG. 8A). VP64-dCas9-VP64-treated iPSCs and ESCs both demonstrated notable expansion potential, averaging 85-fold and 95-fold increase in cell number, respectively, over the 2 weeks after purification. Furthermore, the growth potential of these cells outperformed the PAX7 cDNA overexpressing cells (FIG. 1G, FIG. 8B).

### Example 4

### Characterization of Myogenic Progenitor Cells Derived from Endogenous or Exogenous PAX7 Expression

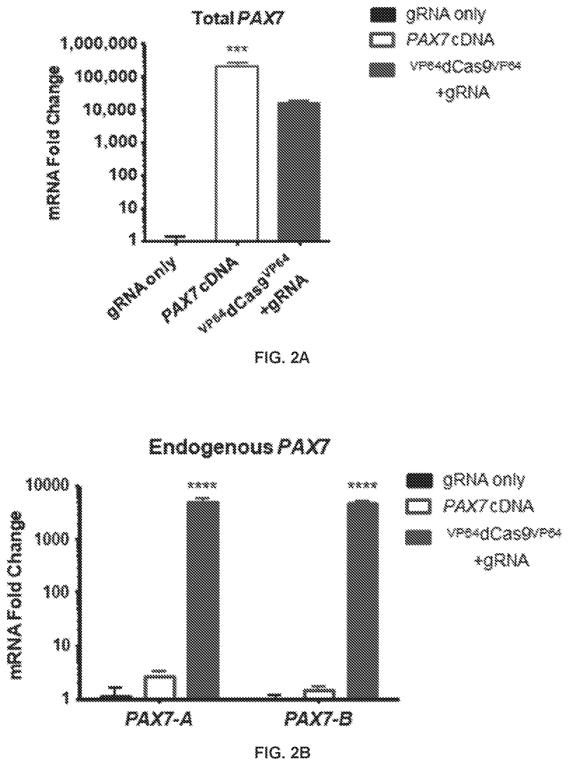

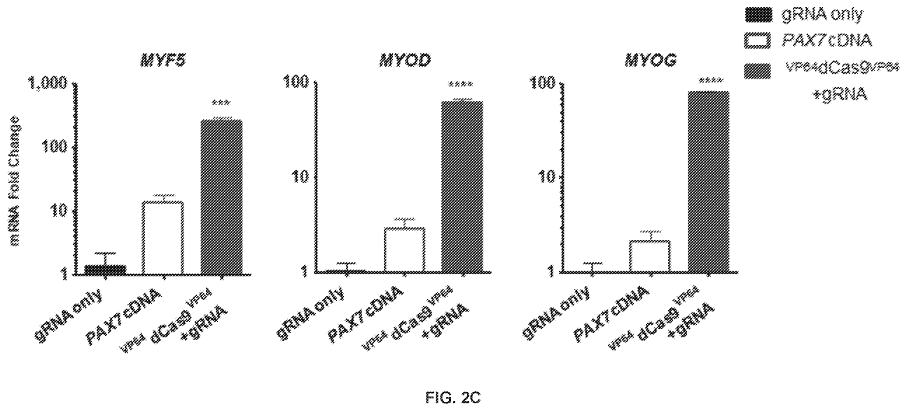

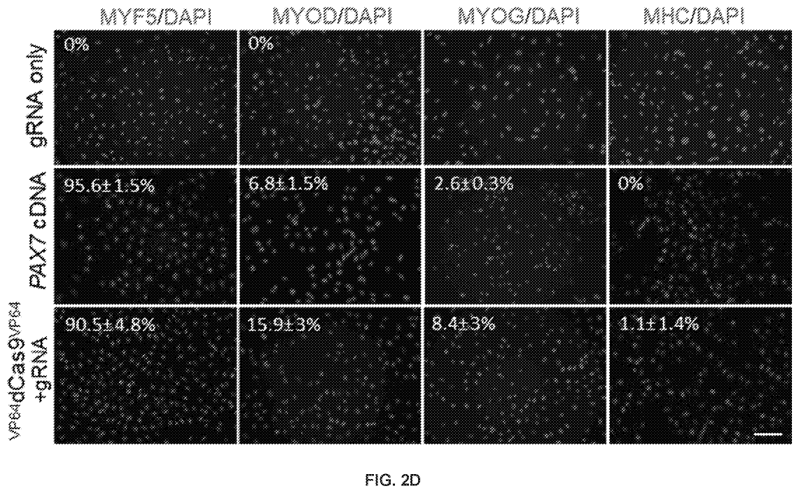

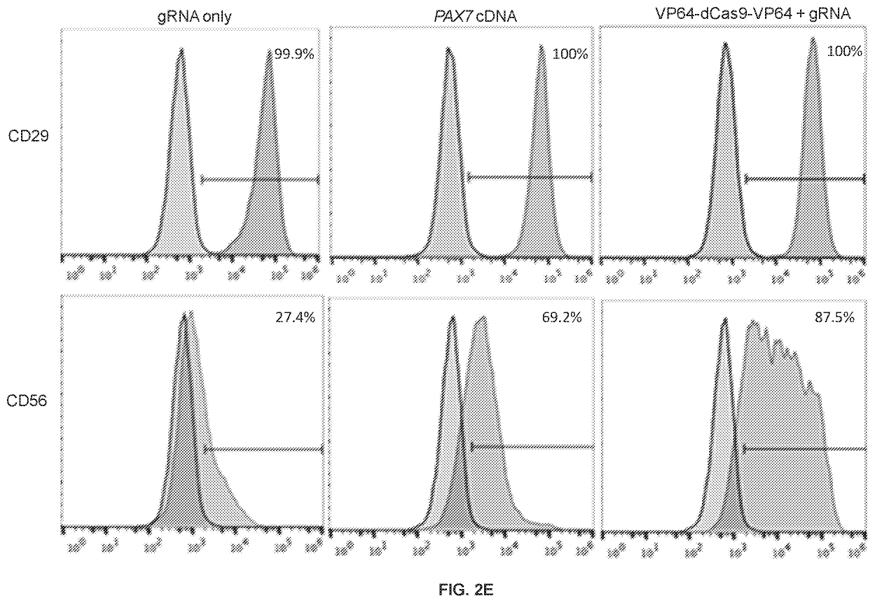

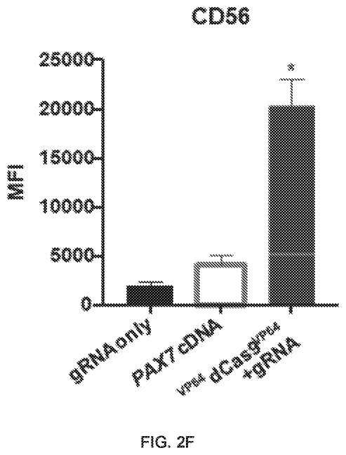

PAX7 mRNA levels were assessed by qRT-PCR during the proliferation phase 5 days after sorting. PAX7 mRNA from the endogenous chromosomal locus could be discriminated from total PAX7 mRNA, made from either the lentivirus or endogenous chromosomal locus, using distinct primer pairs. While overexpression of PAX7 cDNA resulted in more total PAX7 mRNA (FIG. 2A and FIG. 8C), robust detection of any endogenous PAX7 isoform was only observed in VP64-dCas9-VP64-treated cells (FIG. 2B and FIG. 8D). The human PAX7 gene encodes multiple isoforms of which differential sequences have been identified, but unique biological functions remain unclear. Differential transcriptional termination in either exon 8 or exon 9 yield PAX7-A and PAX7-B isoforms, respectively. The differences in the 3′ ends of these transcripts allow for differential detection with unique qRT-PCR primers.

Downstream myogenic regulatory factors MYF5, MYOD, and MYOG were also detected at the mRNA level by qRT-PCR (FIG. 2C, FIG. 8E). At the protein level, the majority of cells in both endogenous and exogenous PAX7-expressing cells co-expressed the activated satellite cell marker, MYF5 (>90%). The myoblast marker, MYOD, was expressed higher in cells expressing endogenous PAX7 compared to exogenous PAX7 cDNA, at 15.9% and 6.8%, respectively. Mature myogenic markers MYOG and Myosin Heavy Chain (MHC) were lowly detectable in some of the cells (FIG. 2D).

Human satellite cells co-express PAX7 with CD29 and CD56 surface markers. At approximately 10 days after sorting, we assessed our MPCs for CD29 and CD56 expression and found 100% of cells in all groups expressed CD29, independent of PAX7 expression. We found CD56 expression was more contingent on PAX7 expression, with only 27.4% of cells expressing CD56 in the gRNA only group, compared to 69.2% and 87.5% of cells in the PAX7 cDNA and VP64-dCas9-VP64-treated groups, respectively (FIG. 2E and FIG. 8F). Assessment of mean fluorescence intensity (MFI) of CD56 staining also revealed the average CD56 expression level per cell was significantly higher in the VP64-dCas9-VP64-treated group (FIG. 2F and FIG. 8G).

### Example 5

### Transplantation of VP64-dCas9-VP64-Generated Myogenic Progenitors into Immunodeficient Mice Demonstrates In Vivo Regenerative Potential

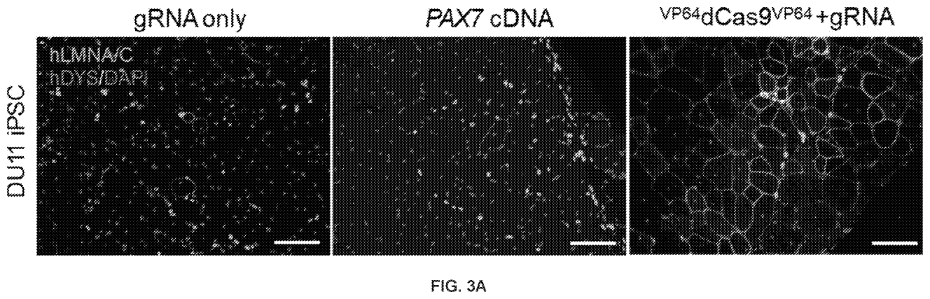

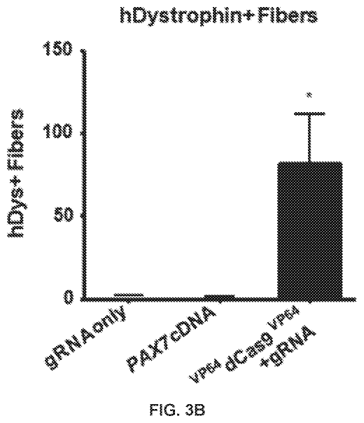

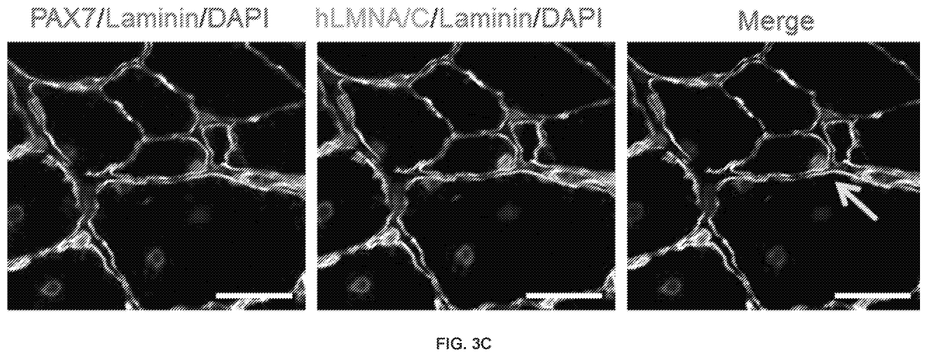

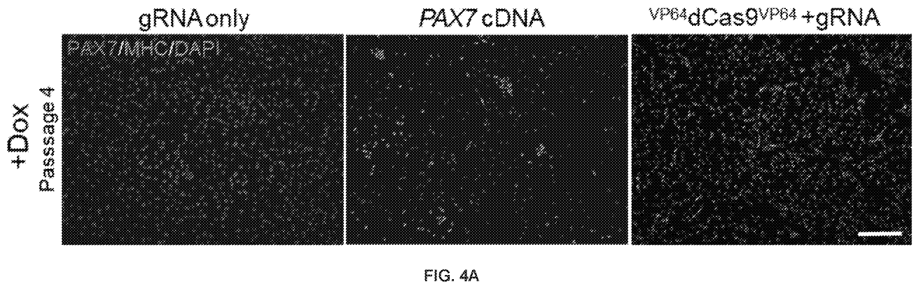

We next determined if MPCs derived from VP64-dCas9-VP64-mediated PAX7 activation possess in vivo regenerative potential. Cells that had been expanded and passaged 3 times post sort were transplanted into the tibialis anterior (TA) of immunodeficient NOD.SCID.gamma (NSG) mice that were pre-injured with barium chloride (BaCl₂) to create a regenerative microenvironment (Hall et al. Sci. Transl. Med. 2010, 2, 57ra83-57ra83). 24 hours after injury, mice were injected with 500,000 cells treated with either gRNA only, PAX7 cDNA overexpression, or VP64-dCas9-VP64-mediated endogenous PAX7 activation. One month after transplantation, muscles were harvested and evaluated for engraftment by immunostaining with human-specific dystrophin and lamin A/C antibodies. Human nuclei were detected by lamin A/C staining in all three conditions; however, only the endogenous PAX7 activated group demonstrated consistent presence of human dystrophin (FIG. 3A and FIG. 8I). The number of human dystrophin+ fibers was quantified across three mice per condition by counting sections with most abundant human dystrophin+ fibers within each sample (FIG. 3B). We also investigated whether transplanted cells could seed the satellite cell niche. Immunostaining for PAX7, human lamin A/C, and laminin was performed to demarcate satellite cells of human origin. PAX7 and human lamin A/C double-positive cells residing under the basal lamina were identified only in muscle transplanted with VP64dCas9VP64-activated MPCs (FIG. 3C, FIG. 8J).

### Example 6

### Induction of Endogenous PAX7 Expression is Sustained after Multiple Passages and Dox Withdrawal

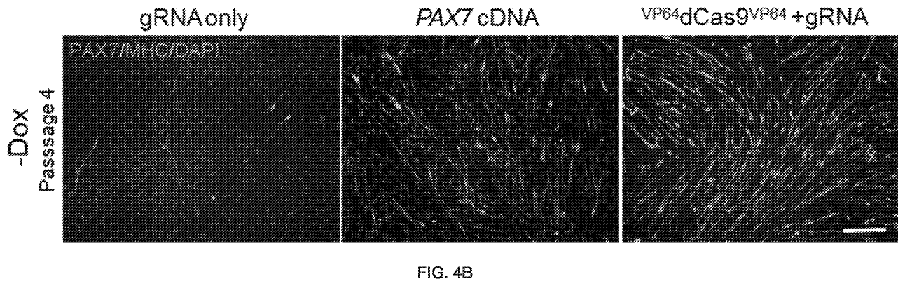

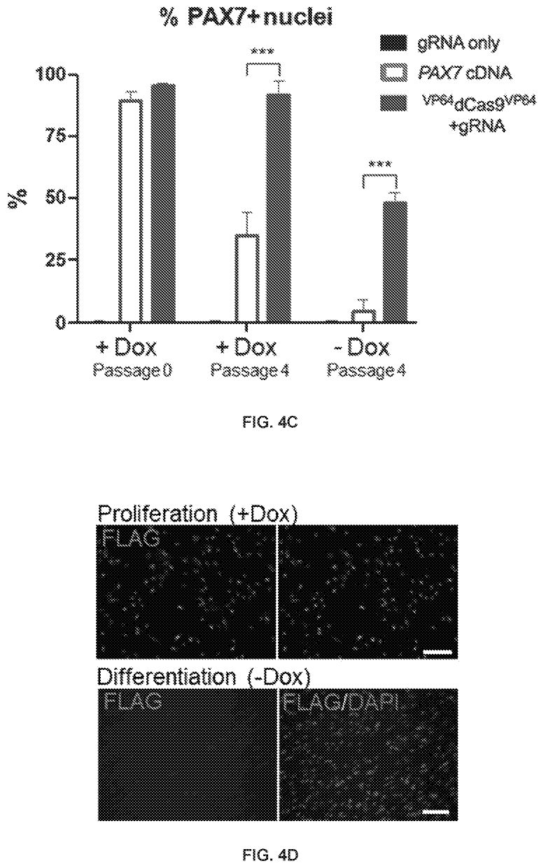

During expansion of sorted cells, we noticed a significant decrease in PAX7+ cells in the cDNA overexpression group after an average of 4 passages spanning an average of 32 days in three independent experiments. Although the initial number of cells expressing PAX7 protein was >90% at five days post sort, quantification of PAX7+ nuclei following approximately 4 passages after initial flow sorting revealed that only a minority of cells (35.8%) expressed PAX7 protein despite maintenance in dox during the expansion period. Conversely, a large majority (93%) of endogenously activated PAX7 cells retained PAX7 protein expression without precocious differentiation across multiple passages (FIG. 4A and FIG. 4C). As indicated by lack of MHC+ cells, depletion of PAX7+ cells in the cDNA overexpression group did not correspond to the adoption of a myogenic fate (FIG. 4A). We postulated this may be due to high levels of PAX7 protein hindering cell proliferation, allowing for cells that have silenced the promoter or contaminating cells from the sort to overtake the cell population. Consistent with this possibility, Pax7 cDNA overexpression has been previously implicated in inducing cell cycle exit without commitment to myogenic differentiation. Interestingly, a previously published study also observed this phenomenon of PAX7 loss over multiple passages when using a tet-inducible PAX7 cDNA overexpression system. That study required amending the serum-free differentiation protocol to media conditions containing highly-mitogenic 20% fetal calf serum to improve retention of PAX7 protein expression in cDNA-overexpressing cells.

Differentiation of premyogenic cells was induced by withdrawing dox when cells reached 100% confluency. Abundant MHC+ myofibers were observed in VP64-dCas9-VP64-treated cells (FIG. 4B, FIG. 8H). Interestingly, 50% of cells remained PAX7+ in these cells in which the endogenous gene had been activated even at 1 week after dox removal, in contrast the PAX7 cDNA-treated cells in which 5.2% were PAX7+ after 1 week without dox (FIG. 4C). Staining for the FLAG epitope confirmed the absence of VP64-dCas9-VP64 in differentiated cells at this time point (FIG. 4D).

### Example 7

### VP64-dCas9-VP64 Leads to Sustained PAX7 Expression and Stable Chromatin Remodeling at Target Locus

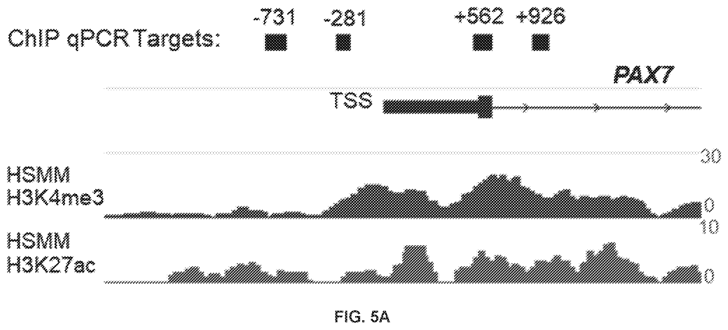

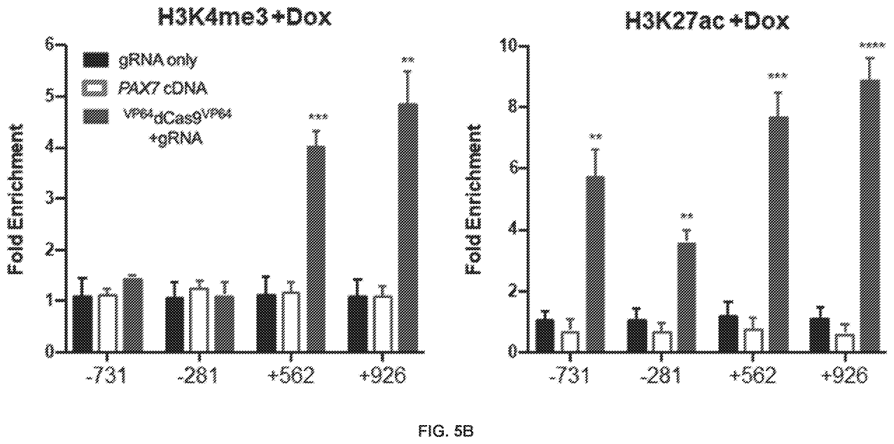

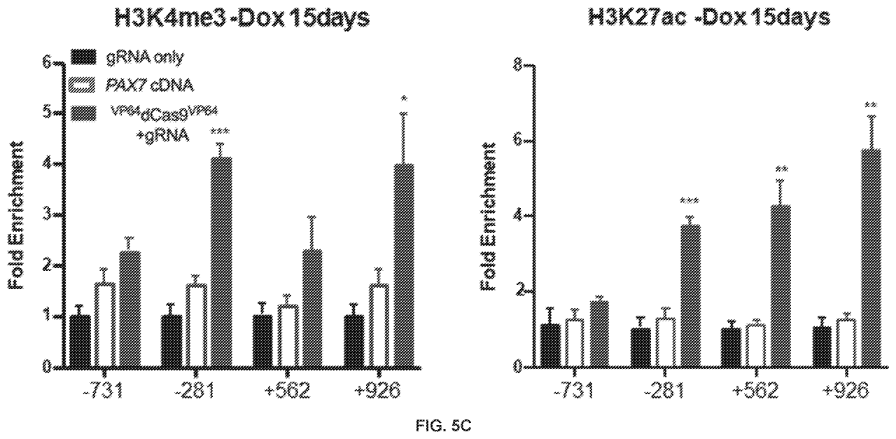

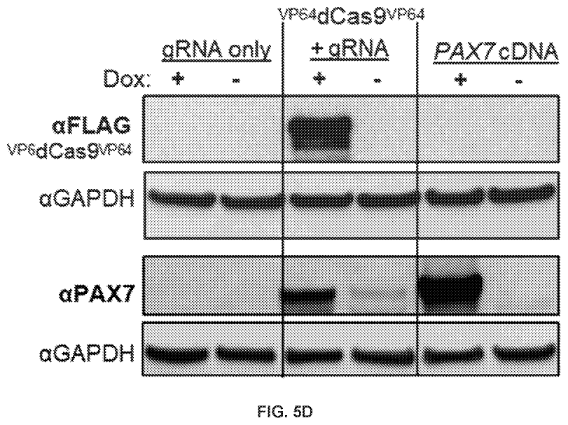

We hypothesized that epigenetic remodeling of the endogenous PAX7 promoter was allowing cells to autonomously upregulate PAX7 without the continued presence of VP64-dCas9-VP64. To investigate this, we performed chromatin immunoprecipitation (ChIP)-qPCR on cells during dox administration and at 15 days after dox withdrawal. Cells were analyzed at day 30 of differentiation for the +dox condition and then expanded and passaged 3 more times over 15 days in the absence of dox. We used ChIP-seq data generated as part of the Encyclopedia of DNA Elements (ENCODE) Project to identify histone modifications enriched at the transcriptionally active PAX7 in human skeletal muscle myoblasts (HSMM), including H3K4me3 and H3K27ac (FIG. 5A). Four qPCR primers were designed to tile regions −731 bp to +926 bp relative to the PAX7 transcription start site (TSS). ChIP qPCR of +dox conditions demonstrated significant enrichment of H3K4me3 and H3K27ac at the endogenous PAX7 locus only in response to VP64-dCas9-VP64 treatment (FIG. 5B). Furthermore, these histone modifications were maintained for 15 days post dox withdrawal (FIG. 5C). To ensure that there was no leaky expression of VP64-dCas9-VP64 after dox removal, we performed a western blot for the FLAG epitope tag and were unable to detect VP64-dCas9-VP64 after 15 days of dox removal (FIG. 5D). Conversely, PAX7 was still detectable by western blot in the absence of VP64-dCas9-VP64, corresponding to the ChIP-qPCR enrichment of active histone marks.

### Example 8

### Identification of Endogenous Vs. Exogenous PAX7-Induced Global Transcriptional Changes

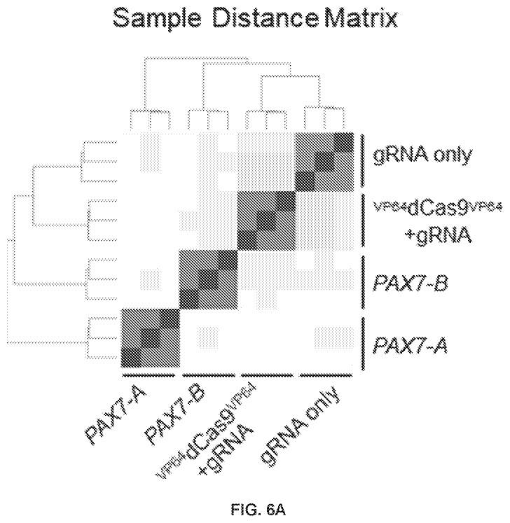

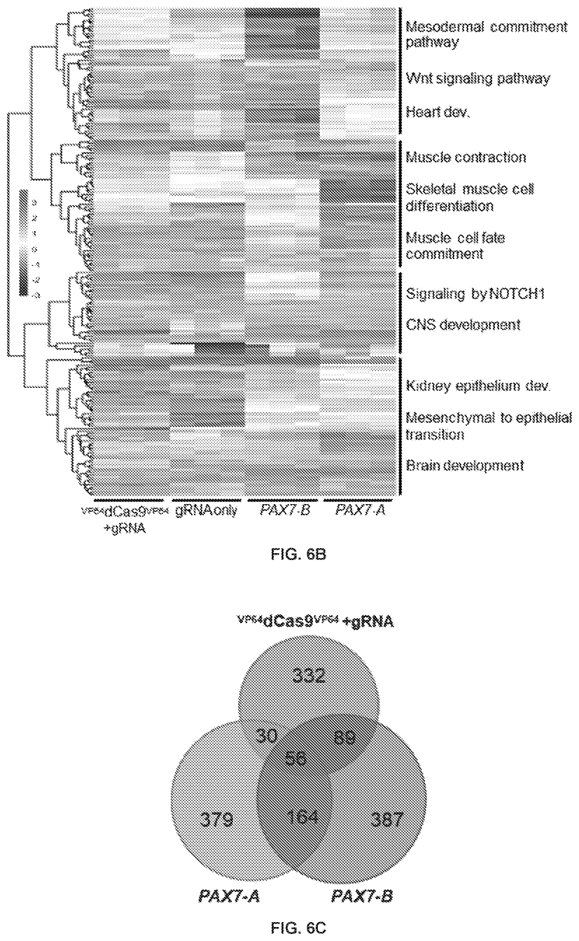

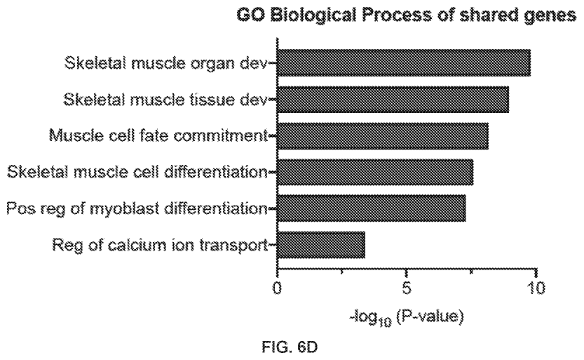

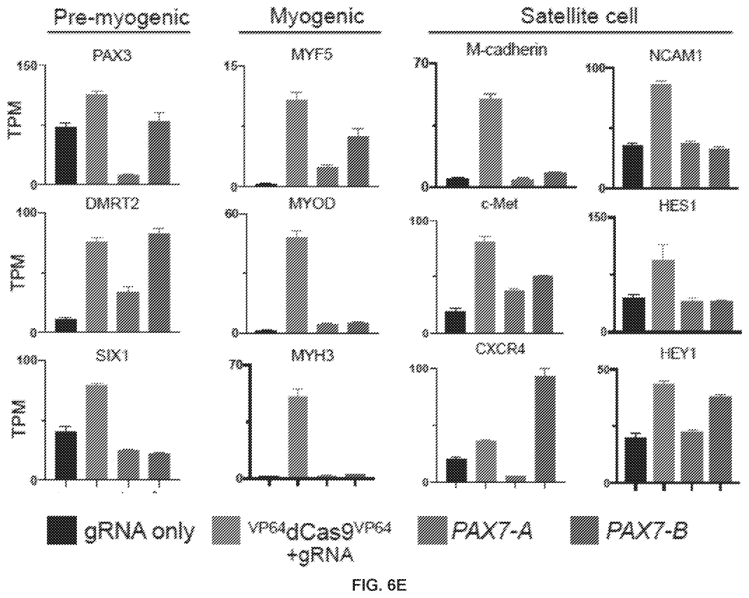

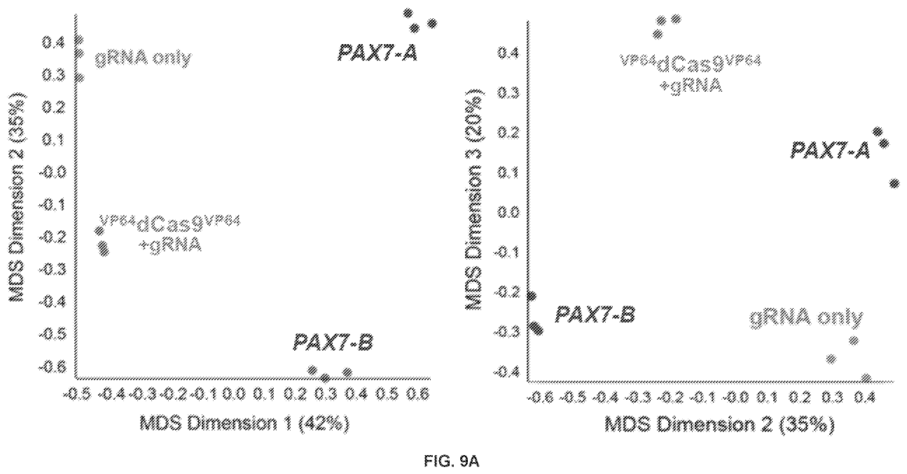

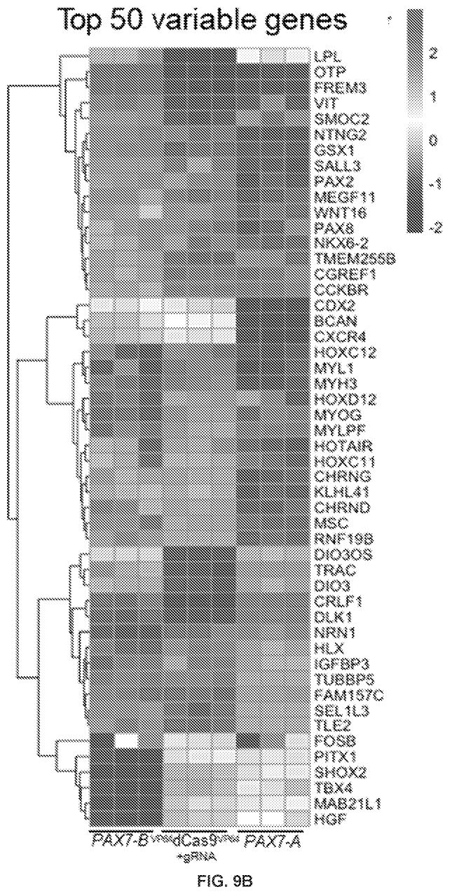

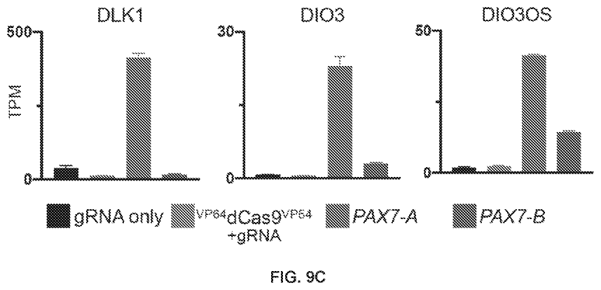

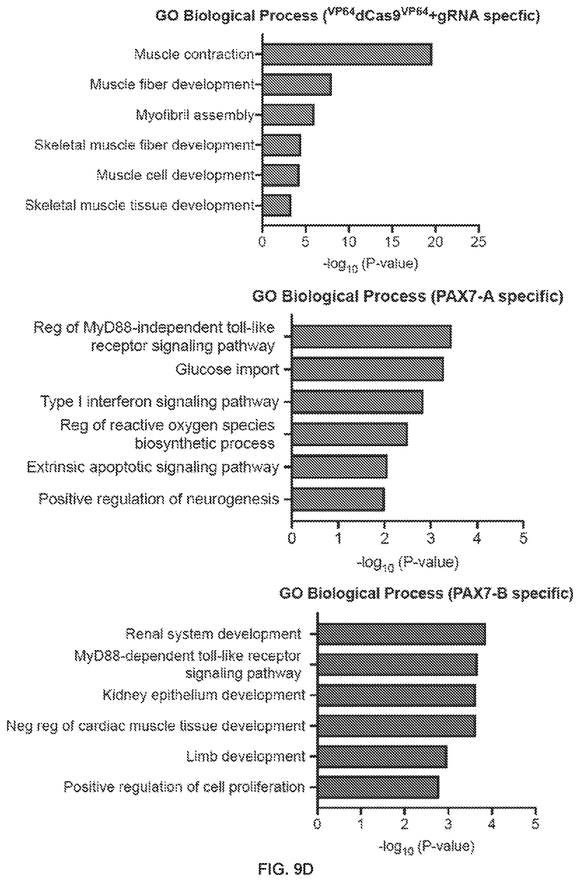

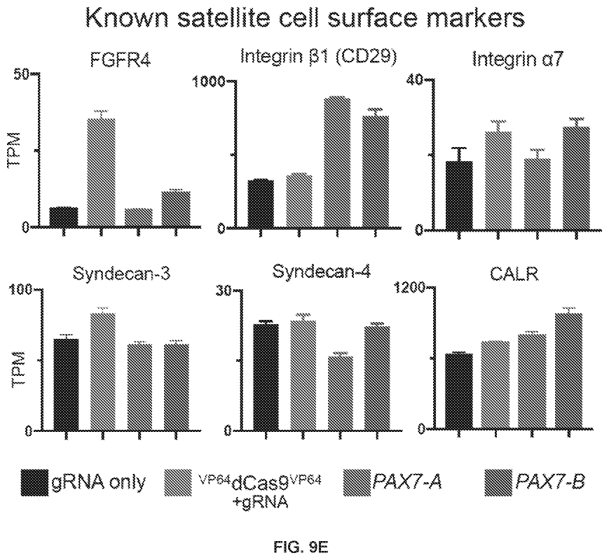

To evaluate the transcriptome-wide gene expression changes induced by endogenous activation of PAX7 compared to exogenous cDNA overexpression, we performed RNA sequencing (RNA-seq) analysis. Differentiated cells that had been treated with either gRNA only, VP64-dCas9-VP64 with gRNA, cDNA encoding PAX7-A isoform, or cDNA encoding PAX7-B isoform were sorted for mCherry expression at day 14 and RNA was extracted for sequencing. We included PAX7-B because it is highly expressed in VP64-dCas9-VP64-treated cells (FIG. 2B), yet little is known of its relationship to PAX7-A. To gauge the variance between the samples, we generated a sample distance matrix of the RNA-seq data (FIG. 6A). This revealed distinct differences between the four treatments, and four unique clusters were readily apparent despite the commonality of induced PAX7 expression in three of the four groups. Multidimensional scaling (MDS) of the top 500 differentially expressed genes also showed divergent clustering of sample groups with PAX7 cDNA overexpression contributing most to variation between transcriptomic profiles (FIG. 9A). We considered the top 200 most variable genes across the 4 groups and submitted lists of gene clusters apparent on the heat map for GO term analysis (FIG. 6B). These analyses revealed general developmental pathways including mesoderm development and WNT signaling pathway genes overexpressed in gRNA only group. Additionally, this group overexpressed genes involved in heart development such as HAND1 and HAND2, which indicates slightly higher propensity of this group to differentiate into cardiac cell lineage. Consistent with this observation, CHIR99021 is also used as the initiator of differentiation of hPSCs into cardiomyocytes.

GO analyses of genes differentially expressed in the VP64-dCas9-VP64 group were strongly related to myogenesis (FIG. 6B and FIG. 9B). Genes represented in this group included embryonic myoblast marker HOXC12, embryonic myosin heavy chain MYH3, as well as other myogenic regulatory factors MYOD and MYOG.

Genes enriched genes following treatment with PAX7-A were associated with CNS development and NOTCH1 signaling pathways. Interestingly, one of the most differentially upregulated genes in this group was DLK1 (FIG. 9B and FIG. 9C), which is required for normal embryonic skeletal muscle development. However, overexpression of DLK1 in vitro inhibits proliferation of satellite cells and induces cell cycle exit and early differentiation. Conversely, Dlk1 knockout increases Pax7+ myogenic progenitor cell proliferation in vitro and enhances post-natal muscle regeneration in vivo. This would suggest that DLK1 is involved in maintaining the balance between quiescence and activation of satellite cells. Furthermore, the specific upregulation of both DLK1 and D103 in these cells (FIG. 9B and FIG. 9C) suggests activity of the DLK1-DIO3 gene cluster. This DLK1-DIO3 locus encodes the largest mammalian megacluster of micro RNAs (miRNA), which is strongly expressed in freshly isolated satellite cells and strongly declined in proliferating satellite cells. This decline of DLK1-DIO3 is concomitant with upregulation of muscle-specific miRNAs, including miR-1, which targets the PAX7 3′ UTR to fine-tune its expression and control satellite cell differentiation. Thus, it is feasible that overexpression of only the PAX7-A isoform results in negative feedback and expression of genes and miRNAs that regulate quiescence.

Genes overexpressed specifically in response to PAX7-B included brain development genes VIT and OTP, as well as other PAX genes, PAX2 and PAX8, which are involved in kidney development. Although PAX7 is not implicated in kidney development, CHIR99021 has been used previously to differentiate hPSCs to a kidney lineage.

Next, we compared each of the three PAX7-expressing groups to the gRNA only group and extracted a list of genes with greater than two-fold change and padj <0.05 after filtering genes with low read counts. We compared these lists of genes and found that the 56 genes shared in all three groups were enriched for GO terms involved in skeletal muscle development (FIG. 6C and FIG. 6D). This suggests that compared to treatment with only the gRNA and 14 days of CHIR-mediated differentiation, all three groups were able to direct hPSCs into the skeletal myogenic program more effectively than the small molecule protocol alone. When individual genes are examined, however, the VP64-dCas9-VP64 group outperforms the other groups in terms of expression of pre-myogenic and myogenic genes (FIG. 6E). Many of the known satellite cell surface markers and genes are also more highly expressed in the VP64-dCas9-VP64 group compared to the other groups, demonstrating more specific and robust commitment to myogenesis and satellite cell differentiation (FIG. 6E and FIG. 9D).

### Example 9

### Discussion

Detailed herein is the utility of CRISPR/Cas9-based transcriptional activators for differentiation of hPSCs into myogenic progenitor cells via targeted activation of the endogenous PAX7 gene. This method may serve as an alternative to the transgene overexpression model that has been previously used for myogenic progenitor cell differentiation. With a minimal small molecule differentiation protocol involving initial paraxial mesodermal differentiation with CHIR99021 and maintenance with FGF2 in serum-free media conditions, it was demonstrated that targeted activation of the endogenous PAX7 gene generates a myogenic progenitor cell population that can be passaged at least 6 times while maintaining PAX7 expression, differentiate readily upon dox withdrawal and subsequent loss of dCas9 activator expression, and engraft into mouse muscle to produce human dystrophin+ fibers while also occupying the satellite cell niche. It was demonstrated that targeting the endogenous PAX7 promoter results in enrichment of H3K4me3 and H3K27ac histone modifications, which was sustained for 15 days after dox removal. Enrichment of these chromatin marks was not observed during overexpression of PAX7 cDNA. Although PAX7 cDNA overexpression from hPSCs has yielded various degrees of engraftment into NSG mice previously, we did not have similar positive engraftment results with PAX7 cDNA overexpression under the conditions used here. However, the prior studies used differentiation protocols that generate embryoid bodies, incorporate additional small molecules, or contain animal serum in the medium and thus, differ from the protocol used in this study. Detailed herein is that activation of the endogenous PAX7 rather than exogenous PAX7 cDNA overexpression increases the efficacy of hPSC differentiation into myogenic progenitor cells with robust growth and differentiation potential, while retaining regenerative properties following transplantation.

Prior studies using exogenous PAX7 cDNA relied on overexpression of only the PAX7-A isoform. However, differential RNA cleavage and polyadenylation yields PAX7-B, which contains a highly conserved paired tail domain and is considered to be the canonical sequence. Both isoforms are expressed in human myogenic cells and orthologs of these PAX7 protein variants are also present in mouse muscle, indicating biological significance for both isoforms. Although distinct functions of these protein variants have not been deciphered, they may play differential roles in myogenesis that may be necessary for proper satellite stem cell function and myogenic differentiation. The RNA-seq analysis demonstrated overlapping myogenic function of cells generated by VP64-dCas9-VP64 endogenous activation or PAX7 cDNA overexpression of either isoforms; however, the VP64-dCas9-VP64 group shared more commonly upregulated genes with PAX7-B than PAX7-A (89 and 30 genes, respectively), indicating a higher degree of similarity, which is also depicted in the sample distance matrix. The dissimilarity between the overexpression of the two cDNAs indicated that they have distinct functions and can influence global gene expression in separate ways. For example, PAX7-B upregulates pre-myogenic genes PAX3, DMRT2, and satellite cell genes CXCR4 and HEY1 more effectively than PAX7-A. Conversely, expression of the DLK1-DIO3 locus that is implicated in satellite cell quiescence is more robust in response to PAX7-A than PAX7-B. VP64-dCas9-VP64-mediated PAX7 induction therefore may allow expression of both isoforms to properly induce myogenesis at levels of expression that are more likely in the physiological range. Furthermore, endogenous activation of PAX7 may preserve the 3′ UTRs, which are binding targets for the many muscle-specific miRNAs that play a role in orchestrating proper muscle development and regeneration.

Although conditional expression of PAX7 in hPSCs via lentiviral transduction may be the most promising approach for generating a homogenous population of engraftable MPCs, integration-free reprogramming may ultimately be used for avoiding undesired consequences of genomic integration of viral vectors. VP64-dCas9-VP64 has been demonstrated to rapidly remodel the epigenetic signature of target loci when gRNAs were transiently delivered to achieve neuronal differentiation. It is demonstrated herein that epigenetic signatures were stably maintained in the absence of VP64-dCas9-VP64. Transient delivery of these targeted transcriptional activators via transfection, electroporation, or nonviral nanoparticle delivery of mRNA/gRNA or purified ribonucleoprotein complexes may offer an alternative to integration-prone methods.

The expansive CRISPR genome engineering toolbox offers many possibilities to manipulate cell fates to improve our understanding of the molecular differences between myoblasts, satellite cells, and MPCs generated from hPSCs. Forced transitioning of cell fate may rely on stochastic factors that have remained largely elusive, but generally include activation of endogenous networks to generate a stable new identity while also opposing epigenetic memory of the old identity. Further investigation of tissue-specific progenitor cell differentiation from pluripotent cells may unveil fundamental guidelines that may inform a revised model for the generation of a well-defined population of cells capable of repopulating the progenitor cell niche long term.

The results detailed herein introduced a novel method for differentiation and expansion of myogenic progenitors from hPSCs by deterministic editing of transcriptional regulation with new genome engineering tools, which may enable new disease modeling and cell therapy in disorders of skeletal muscle regeneration.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure.

Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A guide RNA (gRNA) molecule targeting Pax7, the gRNA comprising a polynucleotide sequence corresponding to at least one of SEQ ID NOs: 1-8 or 69-76, or a variant thereof.

Clause 2. The gRNA of clause 1, wherein the gRNA comprises a crRNA, a tracrRNA, or a combination thereof.

Clause 3. A DNA targeting system for increasing expression of Pax7, the DNA targeting system comprising at least one gRNA that binds and targets a Pax7 gene, a regulatory region of a Pax7 gene, a promoter region of a Pax7 gene, or a portion thereof.

Clause 4. The DNA targeting system of clause 3, wherein the at least one gRNA comprises a polynucleotide sequence corresponding to at least one of SEQ ID NOs: 1-8 or 69-76, or a variant thereof.

Clause 5. The DNA targeting system of clause 3 or 4, wherein the gRNA comprises a crRNA, a tracrRNA, or a combination thereof.

Clause 6. The DNA targeting system of any one of clauses 3-5, further comprising a Clustered Regularly Interspaced Short Palindromic Repeats associated (Cas) protein or a fusion protein, wherein the fusion protein comprises two heterologous polypeptide domains, wherein the first polypeptide domain comprises a Cas protein, a zinc finger protein, or a TALE protein, and the second polypeptide domain has transcription activation activity.

Clause 7. The DNA targeting system of clause 6, wherein the Cas protein comprises a Streptococcus pyogenes Cas9 molecule, or a variant thereof.

Clause 8. The DNA targeting system of clause 6, wherein the fusion protein comprises VP64-dCas9-VP64.

Clause 9. The DNA targeting system of clause 6, wherein the Cas protein comprises a Cas9 that recognizes a Protospacer Adjacent Motif (PAM) of NGG (SEQ ID NO: 31), NGA (SEQ ID NO: 32). NGAN (SEQ ID NO: 33), or NGNG (SEQ ID NO: 34).

Clause 10. An isolated polynucleotide sequence comprising the gRNA molecule of clause 1 or 2.

Clause 11. An isolated polynucleotide sequence encoding the DNA targeting system of any one of clauses 3-9.

Clause 12. A vector comprising the isolated polynucleotide sequence of clause 10 or 11.

Clause 13. A vector encoding the gRNA molecule of clause 1 or 2 and a Clustered Regularly Interspaced Short Palindromic Repeats associated (Cas) protein.

Clause 14. A cell comprising the gRNA of clause 1 or 2, the DNA targeting system of any one of clauses 3-9, the isolated polynucleotide sequence of clause 10 or 11, or the vector of clause 12 or 13, or a combination thereof.

Clause 15. A pharmaceutical composition comprising the gRNA of clause 1 or 2, the DNA targeting system of any one of clauses 3-9, the isolated polynucleotide sequence of clause 10 or 11, the vector of clause 12 or 13, or the cell of clause 14, or a combination thereof.

Clause 16. A method of activating endogenous myogenic transcription factor Pax7 in a cell, the method comprising administering to the cell the gRNA of clause 1 or 2, the DNA targeting system of any one of clauses 3-9, the isolated polynucleotide sequence of clause 10 or 11, or the vector of clause 12 or 13.

Clause 17. A method of differentiating a stem cell into a skeletal muscle progenitor cell, the method comprising administering to the stem cell the gRNA of clause 1 or 2, the DNA targeting system of any one of clauses 3-9, the isolated polynucleotide sequence of clause 10 or 11, or the vector of clause 12 or 13.

Clause 18. The method of clause 17, wherein endogenous expression of Pax7 mRNA is increased in the skeletal muscle progenitor cell.

Clause 19. The method of any one of clauses 17-18, wherein the expression of Myf5, MyoD, MyoG, or a combination thereof, is increased in the skeletal muscle progenitor cell.

Clause 20. The method of any one of clauses 17-19, wherein the stem cell is induced into myogenic differentiation.

Clause 21. The method of any one of clauses 17-20, wherein the skeletal muscle progenitor cell maintains Pax7 expression after at least about 6 passages.

Clause 22. A method of treating a subject in need thereof, the method comprising administering to the subject the cell of clause 14.

Clause 23. The method of clause 22, wherein the level of dystrophin+ fibers in the subject is increased.

Clause 24. The method of clause 22, wherein muscle regeneration in the subject is increased.

### SEQUENCES

| SEQ | | SEQ | |
|---|---|---|---|
| ID | | ID | |
| NO | gRNA seguence | NO | gRNA |
| 1 | ggccggggactcggcggatc | 69 | ggccggggacucggcggauc |
| 2 | tccccggctcgacctcgttt | 70 | uccccggcucgaccucguuu |
| 3 | ccagggcgcaagggagcgg | 71 | ccagggcgcaagggagcgg |
| 4 | tcctccgctcccttgcgccc | 72 | uccuccgcucccuugcgccc |
| 5 | gggggcgcgagtgatcagct | 73 | gggggcgcgagugaucagcu |
| 6 | cgggtttcagggctggacgg | 74 | cggguuucagggcuggacgg |
| 7 | tggtccggagaaagaaggcg | 75 | ugguccggagaaagaaggcg |
| 8 | agcgccagagcgcgagagcg | 76 | agcgccagagcgcgagagcg |

| SEQ ID NO | gRNA target seguence |
|---|---|
| 77 | GATCCGCCGAGTCCCCGGCC |
| 78 | AAACGAGGTCGAGCCGGGGA |
| 79 | CCGCTCCCTTGCGCCCTGG |
| 80 | GGGCGCAAGGGAGCGGAGGA |
| 81 | AGCTGATCACTCGCGCCCCC |
| 82 | CCGTCCAGCCCTGAAACCCG |
| 83 | CGCCTTCTTTCTCCGGACCA |
| 84 | CGCTCTCGCGCTCTGGCGCT |

| Target | Forward Primer (5′-3′) | Reverse Primer (5′-3′) |
|---|---|---|
| GAPDH | gaaggtgaaggtcggagtc | gaagatggtgatgggattc |
| | (SEQ ID NO: 9) | (SEQ ID NO: 10) |
| PAX7 | cagcaagcccagacaggtgg | gcacgcggctaatcgaactc |
| | (SEQ ID NO: 11) | (SEQ ID NO: 12) |
| MYF5 | aatttggggacgagtttgtg | catggtggtggacttcctct |
| | (SEQ ID NO: 13) | (SEQ ID NO: 14) |
| MYOD | agactgccagcactttgcta | gtagctccatatcctggcgg |
| | (SEQ ID NO: 15) | (SEQ ID NO: 16) |
| MYOG | ggtgcccagcgaatgc | gtagctccatatcctggcgg |
| | (SEQ ID NO: 17) | (SEQ ID NO: 18) |
| Endogenous | gctacaaggtggtgtcagggt | gagccatagtacggaagcagag |
| PAX7 | (SEQ ID NO: 19) | (SEQ ID NO: 20) |
| Isoform 1/2 | | |
| Endogenous | tctggccaaaaatgtgagcct | gggtcagttagggttgggc |
| PAX7 | (SEQ ID NO: 21) | (SEQ ID NO: 22) |
| Isoform 3 | | |
| T | tgcttccctgagacccagtt | gatcacttctttcctttgcatcaag |
| | (SEQ ID NO: 23) | (SEQ ID NO: 24) |
| TBX6 | caaccccgcatacacctagt | cgtctcgctccctcttacag |
| | (SEQ ID NO: 25) | (SEQ ID NO: 26) |
| MSGN1 | aacctgcgcgagactttcc | acagctggacagggagaaga |
| | (SEQ ID NO: 27) | (SEQ ID NO: 28) |
| Pax3 | ctcacctcaggtaatgggact | cgtggtggtaggttcagac |
| | (SEQ ID NO: 29) | (SEQ ID NO: 30) |
| PAX7 ChIP | cggggctctgacattacaca | gccagagtccgccctatttc |
| 1, −731 bp | (SEQ ID NO: 61) | (SEQ ID NO: 62 |
| PAX7 ChIP | tattggtcctccgctccctt | gtgagcgcgatctgatagg |
| 2, −289 bp | (SEQ ID NO: 63) | (SEQ ID NO: 64) |
| PAX7 ChIP | ttgccgactttggattcgtc | tccaaagggaatcccgtgc |
| 3, +562 bp | (SEQ ID NO: 65) | (SEQ ID NO: 66) |
| PAX7 ChIP | cgcagggctgaaattctggt | agagccgagaaactgtcagg |
| 4, +926 | (SEQ ID NO: 67) | (SEQ ID NO: 68) |

SEQ ID NO: 31
ngg
SEQ ID NO: 32
nga
SEQ ID NO: 33
ngan
SEQ ID NO: 34
ngng
SEQ ID NO: 35
nggng
SEQ ID NO: 36
nnagaaw
(W = A or T)
SEQ ID NO: 37
naar
(R = A or G)
SEQ ID NO: 38
nngrr
(R = A or G; N can be any nucleotide residue, e.g., any of A, G, C, or T)
SEQ ID NO: 39
nngrrn
(R = A or G; N can be any nucleotide residue, e.g., any of A, G, C, or T)
SEQ ID NO: 40
nngrrt
(R = A or G; N can be any nucleotide residue, e.g., any of A, G, C, or T)
SEQ ID NO: 41
nngrrv
(R = A or G; N can be any nucleotide residue, e.g., any of A, G, C, or T)
codon optimized polynucleotide encoding S. pyogenes Cas9
SEQ ID NO: 42
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcdgttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tcttgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagcgaga aaatattgtg
atcgagatgg cccgcgaaaa ccagaccaca cagaagggcc agaaaaatag tagagagcgg
atgaagagga tcgaggaggg catcdaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tgggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga
aacacggatcgacctctctc aactgggcgg cgactag
Amino acid seguence of Streptococcus pyogenes Cas9
SEQ ID NO: 43
MDKKYSIGLDIGTNSVGWAVITDEYKVPSKKFKVLGNTDRHSIKKNLIGALLFDSGETASATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIKLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMMFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
codon optimized nucleic acid seguence encoding S. aureus Cas9
SEQ ID NO: 44
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
codon optimized nucleic acid seguence encoding S. aureus Cas9
SEQ ID NO: 45
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccgc ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tcttcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgatc
aacaagagcc cggaaaagct gctgatgtac caccacgacc cccagaccta ccagaaactg
aagctgatta tggaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
codon optimized nucleic acid seguence encoding S. aureus Cas9
SEQ ID NO: 46
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctcca tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tcttcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tgtgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcatc aagaaggga
codon optimized nucleic acid seguence encoding S. aureus Cas9
SEQ ID NO: 47
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
codon optimized nucleic acid seguence encoding S. aureus Cas9
SEQ ID NO: 48
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attLLccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac cgggaaagag
aacgcaaagt acctgattga aaaaatcaag ctgcacgata tgcaggaggg aaagtgtctg
tattctctgg aggccatccc cctggaggac ctgctgaaca atccaLtcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatggrga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgcctc taagactcag agtatcaaaa agtactcaac cgacattctg
ggaaacctgt atgaggtgaa gagcaaaaag caccctcaga ttatcaaaaa gggctaagaa
ttc
Amino acid seguence of Staphylococcus aureus Cas9
SEQ ID NO: 49
MKRNYILGLDIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVQKAYHQLDQSFIDTYIDL
LETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKE
IIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDVKVKSINGGF
TSFLRRKWKFKKERNKGYKHHAEDALIIANADFIFKEWKKLDKAKKVMENQMFEEKQAESMPEIETEQ
EYKEIFITPHQIKHIKDFKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
Nucleic acid seguence encoding D10A mutant of S. aureus Cas9
SEQ ID NO: 50
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata tctttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
Nucleic acid seguence encoding N580A mutant of S. aureus Cas9
SEQ ID NO: 51
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt ccagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
codon optimized nucleic acid seguence encoding S. aureus Cas9
SEQ ID NO: 52
atggccccaaagaagaagcgcaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagaggagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
codon optimized nucleic acid sequence encoding S. aureus Cas9
SEQ ID NO: 53
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacatgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatccaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
Streptococcus pyogenes Cas9 (with D10A, H849A)
SEQ ID NO: 54
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEKHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMKTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
Vector (pDO242) encoding codon optimized nucleic acid sequence
encoding S. aureus Cas9
SEQ ID NO: 55
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatqgtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACGATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGAGATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATGAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcqgtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatqgttatgqcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcqgggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac
SEQ ID NO: 56
tttn
(N can be any nucleotide residue, e.g., any of A, G, C, or T)
VP64-dCas9-VP64 protein
SEQ ID NO: 57
RADALDDFDLDMLGSDALDDFDLDMLGSDALDDFDLDMLGSDALDDFDLDMVNPKKKRKVGRGMDKKY
SIGLAIGTNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
KKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKPMLASAGELQKGNELALP
SKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRIDLSQL
GGDSRADPKKKRKVASRADALDDFDLDMLGSDALDDFDLDMLGSDALDDFDLDMLGSDALDDFDLDML
I
VP64-dCas9-VP64 DNA
SEQ ID NO: 58
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatggttaaccccaagaagaagaggaaggtgggccgcggaatggacaagaagtac
tccattgggctcgccatcggcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctgctacctgcaggagatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagggggacctgaacccagacaacagcgatgtcgacaaactctttatcc
aactggttcagacttacaatcagcttttcgaagagaacccgatcaacgcatccqgagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatqgctacgccggatacattgacggcggagcaag
ccaggaggaattttacaaatttattaagcccatcttggaaaaaatggacggcaccgaggagctgctgg
taaagcttaacagagaagatctgttgcgcaaacagcgcactttcgacaatggaagcatcccccaccag
attcacctgggcgaactgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgagtacctcacagtttataacgagctcaccaagg
tcaaatacgtcacagaagggatgagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcacccttacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggatttccttaagccc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaagg
cataagcccgagaatatcgttatcgagatggcccgagagaaccaaactacccagaagggacagaagaa
cagtagggaaaggatgaagaggattgaagagggtataaaagaactggggtcccaaatccttaaggaac
acccagttgaaaacacccagcttcagaatgagaagctctacctgtactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggcggcagctgctgaacgcc
aaactgatcacacaacggaagttcgataatctgactaaggctgaacgaggtggcctgtctgagttgga
taaagccggcttcatcaaaaggcagcttgttgagacacgccagatcaccaagcacgtggcccaaattc
tcgattcacgcatgaacaccaagtacgatgaaaatgacaaactgattcgagaggtgaaagttattact
ctgaagtctaagctggtctcagatttcagaaaggactttcagttttataaggtaagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagcct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cgacaagctgatcgcacgcaaaaaagattgggaccccaagaaatacggcggattcgattctcctacag
tcgcttacagtgtactggttgtggccaaagtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctagtgcgggcgagctgcagaaaggtaacgagctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
ttccgatctcgacatgctgggttctgatgccctcgatgactttgacctggatatgttgggaagcgacg
cattggatgactttgatctggacatgctcggctccgatgctctggacgatttcgatctcgatatgtta
atc
Human p300 (with L553M mutation) protein
SEQ ID NO: 59
MAENVVEPGPPSAKRFKLSSPALSASASDGTDFGSLFDLEHDLPDELINSTELGLTNGGDINQLQTSL
GMVQDAASKHKQLSELLRSGSSPNLNMGVGGPGQVMASQAQQSSPGLGLINSMVKSPMTQAGLTSPNM
GMGTSGPNQGPTQSTGMMNSPVNQPAMGMNTGMNAGMNPGMLAAGNGQGIMPNQVMNGSIGAGRGRQN
MQYPNPGMGSAGNLLTEPLQQGSPQMGGQTGLRGPQPLKMGMMNNPNPYGSPYTQNPGQQIGASGLGL
QIQTKTVLSNNLSPFAMDKKAVPGGGMPNMGQQPAPQVQQPGLVTPVAQGMGSGAHTADPEKRKLIQQ
QLVLLLHAHKCQRREQANGEVRQCNLPHCRTMKNVLNHMTHCQSGKSCQVAHCASSRQIISHWKNCTR
HDCPVCLPLKNAGDKRNQQPILTGAPVGLGNPSSLGVGQQSAPNLSTVSQIDPSSIERAYAALGLPYQ
VNQMPTQPQVQAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMENLVAYA
RKVEGDMYESANNRAEYYHLLAEKIYKIQKELEEKRRTRLQKQNMLPNAAGMVPVSMNPGPNMGQPQP
GMTSNGPLPDPSMIRGSVPNQMMPRITPQSGLNQFGQMSMAQPPIVPRQTPPLQHHGQLAQPGALNPP
MGYGPRMQQPSNQGQFLPQTQFPSQGMNVTNIPLAPSSGQAPVSQAQMSSSSCPVNSPIMPPGSQGSH
IHCPQLPQPALHQNSPSPVPSRTPTPHHTPPSIGAQQPPATTIPAPVPTPPAMPPGPQSQALHPPPRQ
TPTPPTTQLPQQVQPSLPAAPSADQPQQQPRSQQSTAASVPTPTAPLLPPQPATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPESLPFRQPVDPQLLGIPD
YFDIVKSPMDLSTIKRKLDTGQYQEPWQYVDDIWLMFNNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDDPSQPQT
TINKEQFSKRKNDTLDPELFVECTECGRKMHQICVLHHEIIWPAGFVCDGCLKKSARTRKENKFSAKR
LPSTRLGTFLENRVNDFLRRQNHPESGEVTVRVVHASDKTVEVKPGMKARFVDSGEMAESFPYRTKAL
FAFEEIDGVDLCFFGMHVQEYGSDCPPPNQRRVYISYLDSVHFFRPKCLRTAVYHEILIGYLEYVKKL
GYTTGHIWACPPSEGDDYIFHCHPPDQKIPKPKRLQEWYKKMLDKAVSERIVHDYKDIFKQATEDRLT
SAKELPYFEGDFWPNVLEESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTMCMLVELHTQSQDRFVYTCNECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
KGCKRKTNGGCPICKQLIALCCYHAKHCQENKCPVPFCLNIKQKLRQQQLQHRLQQAQMLRRRMASMQ
RTGVVGQQQGLPSPTPATPTTPTGQQPTTPQTPQPTSQPQPTPPNSMPPYLPRTQAAGPVSQGKAAGQ
VTPPTPPQTAQPPLPGPPPAAVEMAMQIQRAAETQRQMAHVQIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVLSILHANPQLLAAFIKQRAAKYANSNPQPIPGQPGMPQ
GQPGLQPPTMPGQQGVHSKPAMQNMNPMQAGVQRAGLPQQQPQQQLQPPMGGMSPQAQQMNMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPNPMSPQQHMLPNQAQSPHLQGQQIPNSLSNQVRSPQP
VPSPRPQSQPPHSSPSPRMQPQPSPRHVSPQTSSPHPGLVAAQANPMEQGHFASPDQNSMLSQLASNP
GMANLHGASATDLGLSTDNSDLNSNLSQSTLDIH
Human p300 Core Effector protein (aa 1048-1664 of SEQ ID NO: 59)
SEQ ID NO: 60
IFKPEELRQALMPTLEALYRQDPESLPFRQPVDPQLLGIPDYFDIVKSPMDLSTIKRKLDTGQYQEPW
QYVDDIWLMFNNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDDPSQPQTTINKEQFSKRKNDTLDPELFVECTECG
RKMHQICVLHHEIIWPAGFVCDGCLKKSARTRKENKFSAKRLPSTRLGTFLENRVNDFLRRQNHPESG
EVTVRVVHASDKTVEVKPGMKARFVDSGEMAESFPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDFWPNVLEESIKELEQE
EEERKREENTSNESTDVTKGDSKNAKKKNNKKTSKNKSSLSRGNKKKPGMPNVSNDLSQKLYATMEKH
KEVFFVIRLIAGPAANSLPPIVDPDPLIPCDLMDGRDAFLTLARDKHLEFSSLRRAQWSTMCMLVELH
TQSQD
Polynucleotide sequence of a gRNA scaffold
SEQ ID NO: 85
gttttagagctagaaatagcaagttaaaataaggctagtccgttatcaacttgaaaaagtgg
caccgagtcggtgcttttttt

1. A guide RNA (gRNA) molecule targeting Pax7, the gRNA comprising a polynucleotide sequence corresponding to at least one of SEQ ID NOs: 1-8 or 69-76, or a variant thereof.

2. The gRNA of claim 1, wherein the gRNA comprises a crRNA, a tracrRNA, or a combination thereof.

3. A DNA targeting system for increasing expression of Pax7, the DNA targeting system comprising at least one gRNA that binds and targets a Pax7 gene, a regulatory region of a Pax7 gene, a promoter region of a Pax7 gene, or a portion thereof.

4. The DNA targeting system of claim 3, wherein the at least one gRNA comprises a polynucleotide sequence corresponding to at least one of SEQ ID NOs: 1-8 or 69-76, or a variant thereof.

5. The DNA targeting system of claim 3 or 4, wherein the gRNA comprises a crRNA, a tracrRNA, or a combination thereof.

6. The DNA targeting system of any one of claims 3-5, further comprising a Clustered Regularly Interspaced Short Palindromic Repeats associated (Cas) protein or a fusion protein,
wherein the fusion protein comprises two heterologous polypeptide domains, wherein the first polypeptide domain comprises a Cas protein, a zinc finger protein, or a TALE protein, and the second polypeptide domain has transcription activation activity.

7. The DNA targeting system of claim 6, wherein the Cas protein comprises a Streptococcus pyogenes Cas9 molecule, or a variant thereof.

8. The DNA targeting system of claim 6, wherein the fusion protein comprises VP64-dCas9-VP64.

9. The DNA targeting system of claim 6, wherein the Cas protein comprises a Cas9 that recognizes a Protospacer Adjacent Motif (PAM) of NGG (SEQ ID NO: 31), NGA (SEQ ID NO: 32), NGAN (SEQ ID NO: 33), or NGNG (SEQ ID NO: 34).

10. An isolated polynucleotide sequence comprising the gRNA molecule of claim 1 or 2.

11. An isolated polynucleotide sequence encoding the DNA targeting system of any one of claims 3-9.

12. A vector comprising the isolated polynucleotide sequence of claim 10 or 11.

13. A vector encoding the gRNA molecule of claim 1 or 2 and a Clustered Regularly Interspaced Short Palindromic Repeats associated (Cas) protein.

14. A cell comprising the gRNA of claim 1 or 2, the DNA targeting system of any one of claims 3-9, the isolated polynucleotide sequence of claim 10 or 11, or the vector of claim 12 or 13, or a combination thereof.

15. A pharmaceutical composition comprising the gRNA of claim 1 or 2, the DNA targeting system of any one of claims 3-9, the isolated polynucleotide sequence of claim 10 or 11, the vector of claim 12 or 13, or the cell of claim 14, or a combination thereof.

16. A method of activating endogenous myogenic transcription factor Pax7 in a cell, the method comprising administering to the cell the gRNA of claim 1 or 2, the DNA targeting system of any one of claims 3-9, the isolated polynucleotide sequence of claim 10 or 11, or the vector of claim 12 or 13.

17. A method of differentiating a stem cell into a skeletal muscle progenitor cell, the method comprising administering to the stem cell the gRNA of claim 1 or 2, the DNA targeting system of any one of claims 3-9, the isolated polynucleotide sequence of claim 10 or 11, or the vector of claim 12 or 13.

18. The method of claim 17, wherein endogenous expression of Pax7 mRNA is increased in the skeletal muscle progenitor cell.

19. The method of any one of claims 17-18, wherein the expression of Myf5, MyoD, MyoG, or a combination thereof, is increased in the skeletal muscle progenitor cell.

20. The method of any one of claims 17-19, wherein the stem cell is induced into myogenic differentiation.

21. The method of any one of claims 17-20, wherein the skeletal muscle progenitor cell maintains Pax7 expression after at least about 6 passages.

22. A method of treating a subject in need thereof, the method comprising administering to the subject the cell of claim 14.

23. The method of claim 22, wherein the level of dystrophin+ fibers in the subject is increased.

24. The method of claim 22 or 23, wherein muscle regeneration in the subject is increased.
